SEQUENCE LISTING (12 sequences)

SEQ ID NO: 1 - protein, 1205 aa, Bos taurus
  SOURCE 1..1205: /mol_type=“protein” /organism=“Bos taurus”
MGNLKSVGQEPGPPCGLGLGLGLGLCGKQGPASPAPEPSRAPAPATPHAPDHSPAPNSPTLTRPPEGPKFPRVKNWELGSITYDTLCAQSQQDGPCTPRCCLGSLVLPRKLQTRPSPGPPPAEQLLSQARDFINQYYSSIKRSGSQAHEERLQEVEAEVASTGTYHLRESELVFGAKQAWRNAPRCVGRIQWGKLQVFDARDCSSAQEMFTYICNHIKYATNRGNLRSAITVFPQRAPGRGDFRIWNSQLVRYAGYRQQDGSVRGDPANVEITELCIQHGWTPGNGRFDVLPLLLQAPDEAPELFVLPPELVLEVPLEHPTLEWFAALGLRWYALPAVSNMLLEIGGLEFSAAPFSGWYMSTEIGTRNLCDPHRYNILEDVAVCMDLDTRTTSSLWKDKAAVEINLAVLHSFQLAKVTIVDHHAATVSFMKHLDNEQKARGGCPADWAWIVPPISGSLTPVFHQEMVNYILSPAFRYQPDPWKGSATKGAGITRKKTFKEVANAVKISASLMGTLMAKRVKATILYASETGRAQSYAQQLGRLFRKAFDPRVLCMDEYDVVSLEHEALVLVVTSTFGNGDPPENGESFAAALMEMSGPYNSSPRPEQHKSYKIRFNSVSCSDPLVSSWRRKRKESSNTDSAGALGTLRFCVFGLGSRAYPHFCAFARAVDTRLEELGGERLLQLGQGDELCGQEEAFRGWAKAAFQASCETFCVGEEAKAAAQDIFSPKRSWKRQRYRLSTQAEGLQLLPGLIHVHRRKMFQATVLSVENLQSSKSTRATILVRLDTAGQEGLQYQPGDHIGICPPNRPGLVEALLSRVEDPPPPTESVAVEQLEKGSPGGPPPSWVRDPRLPPCTLRQALTFFLDITSPPSPRLLRLLSTLAEEPSEQQELETLSQDPRRYEEWKWFRCPTLLEVLEQFPSVALPAPLLLTQLPLLQPRYYSVSSAPNAHPGEVHLTVAVLAYRTQDGLGPLHYGVCSTWLSQLKTGDPVPCFIRGAPSFRLPPDPYVPCILVGPGTGIAPFRGFWQERLHDIESKGLQPAPMTLVFGCRCSQLDHLYRDEVQDAQERGVFGRVLTAFSREPDSPKTYVQDILRTELAAEVHRVLCLERGHMFVCGDVTMATSVLQTVQRILATEGDMELDEAGDVIGVLRDQQRYHEDIFGLTLRTQEVTSRIRTQSFSLQERHLRGAVPWAFDPPGPDTPGP

SEQ ID NO: 2 - protein, 1203 aa, Homo sapiens
  SOURCE 1..1203: /mol_type=“protein” /organism=“Homo sapiens”
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDEPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

SEQ ID NO: 3 - protein, 4 aa, Bos taurus
  SOURCE 1..4: /mol_type=“protein” /organism=“Bos taurus”
PWAF

SEQ ID NO: 4 - protein, 4 aa, Bos taurus
  SOURCE 1..4: /mol_type=“protein” /organism=“Bos taurus”
GAVP

SEQ ID NO: 5 - protein, 21 aa, Bos taurus
  SOURCE 1..21: /mol_type=“protein” /organism=“Bos taurus”
RHLRGAVPWAFDPPGPDTPGP

SEQ ID NO: 6 - protein, 12 aa, Bos taurus
  SOURCE 1..12: /mol_type=“protein” /organism=“Bos taurus”
AFDPPGPDTPGP

SEQ ID NO: 7 - protein, 11 aa, Bos taurus
  SOURCE 1..11: /mol_type=“protein” /organism=“Bos taurus”
HLRGAVPWAFD

SEQ ID NO: 8 - protein, 20 aa, Bos taurus
  SOURCE 1..20: /mol_type=“protein” /organism=“Bos taurus”
HLRGAVPWAFDPPGPDTPGP

SEQ ID NO: 9 - protein, 92 aa, Bos taurus
  SOURCE 1..92: /mol_type=“protein” /organism=“Bos taurus”
MSELEKAVVALIDVFHQYSGREGDKHKLKKSELKELINNELSHFLEEIKEQEVVDKVMETLDSDGDGECDFQEFMAFVAMITTACHEFFEHE

SEQ ID NO: 10 - protein, 92 aa, Homo sapiens
  SOURCE 1..92: /mol_type=“protein” /organism=“Homo sapiens”
MSELEKAMVALIDVFHQYSGREGDKHKLKKSELKELINNELSHFLEEIKEQEVVDKVMETLDNDGDGECDFQEFMAFVAMVTTACHEFFEHE

SEQ ID NO: 11 - protein, 94 aa, Bos taurus
  SOURCE 1..94: /mol_type=“protein” /organism=“Bos taurus”
MGSELETAMETLINVFHAHSGKEGDKYKLSKKELKELLQTELSGFLDAQKDVDAVDKVMKELDENGDGEVDFQEYVVLVAALTVACNNFFWENS

SEQ ID NO: 12 - protein, 94 aa, Bos taurus
  SOURCE 1..94: /mol_type=“protein” /organism=“Bos taurus”
MGSELETAMETLINVFHAHSGKEGDKYKLSKKELKELLQTELSGFLDAQKDADAVDKVMKELDENGDGEVDFQEYVVLVAALTVACNNFFWENS

(19) United States
(12) Patent Application Publication
(10) Pub. No.: US 2013/0058981 A1
(43) Pub. Date: Mar. 7, 2013

(54) Combination pharmaceutical compositions and method of treatment of vertigo, kinetosis and vegetative-vascular dystonia

(71) Applicant: Oleg Iliich Epshtein, Moscow (RU)

(72) Inventor: Oleg Iliich Epshtein, Moscow (RU)

(21) Appl. No.: 13/135,887

(22) Filed: Jul. 15, 2011

(30) Foreign Application Priority Data
Jul. 21, 2010 (RU) 2010130353
Jul. 21, 2010 (RU) 2010130356
Jul. 1, 2011 (RU) 2011127052
Jul. 1, 2011 (RU) 2011127058

(51) Int. Cl. A61K 39/395; A61P 1/08; A61P 25/00; A61K 9/00

(52) U.S. Cl. 424/400; 424/139.1

(57) ABSTRACT

The present invention relates to combination pharmaceutical compositions comprising an activated-potentiated form of an antibody to endothelial NO synthase and activated potentiated form of an antibody to brain-specific protein S-100 and its use for the treatment of vegetative-vascular dystonia (VVD) and symptoms thereof.

### FIELD

The present invention relates to combination pharmaceutical compositions comprising an activated-potentiated form of an antibody to NO synthase and activated potentiated form of an antibody to protein S-100 and its use for the treatment of vertigo of various genesis, kinetosis and vegetative-vascular dystonia.

### BACKGROUND

Vegetative-vascular dystonia (VVD) (synonyms: neurocirculatory dystonia, neurocirculatory asthenia, psychovegetative syndrome, vegetative neurosis, syndrome of vegetative dysfunction syndrome (VDS); and polyetiologic syndrome characterized by dysfunction of vegetative (autonomous) nervous system (VNS) are functional (that is non-organic) disorders that affect most of the systems of the body in an organism (mainly cardiovascular system). The main clinical peculiarity of subjects with VVD is the presence of numerous complaints and a variety symptoms and syndromes caused by peculiarities of the pathogenesis involved in the process of hypothalamic structures. The most frequent symptoms of VVD are: cardialgia, asthenia, neurotic disorders, headache, sleep disturbance, vertigo, respiratory disorders, tachycardia, extremity coldness, vegetative-vascular paroxysms, arm trembling, internal tremor, cardiophobia, myalgia, joint pains, tissue swelling, heart intermittence, feeling of heat on face, low-grade pyrexia, and fainting.

Vegetative symptoms that are evident in disorder of regulation of vegetative-vascular, respiratory and other systems of organism can also be components of a number of disease states, for example: hypertensive disease, endocrine disorders, chronic ischemic heart diseases etc. Thus, vegetative-vascular dystonia and neurocirculatory dystonia can be ascertained in subjects on the basis of a complex of symptoms that is typical for somatoform dysfunction of vegetative nervous system.

As part of the complex of symptoms of vegetative-vascular dystonia, one can distinguish a separately isolated cerebrovascular disorders which is characterized by headaches, vertigos, buzzing in head and ears, weakness of vestibular apparatus, tendency to faint and kinetosis. At the heart of its development are cerebral angiodystonia, the pathogenetic basis of which is disregulation of vascular tone of the brain, hypertonic, hypotonic or mixed character.

Kinetosis (synonyms: motion sickness, sea sickness, air sickness, car sickness etc.) is a disease of movement (Greek: kynesis—motion) that appears on action of the body that are more or less long-lasting and of variable accelerations. Disorders of coordination of movements, vertigo, nausea, vomiting, pallor, cold sweat, reduction of blood pressure, infrequent heartbeats are typical for kinetosis. In severe cases, depression, asthenias, disorders of lucidity are possible. However after cessation of accelerations kinetosis symptoms disappear. Due to the fact that at the moment of motion sickness different receptors of vestibular apparatus become inflamed in turn, the cerebellum receives impulses causing changes in the tone of various groups of muscles of the neck, the back, and the extremities, hence giving rise to the appearance of asymmetry of muscle tone and in coordination of muscle movements. Manifestations of kinetosis are more expressed within persons with hyperexcitability of sympathetic or parasympathetic parts of nervous system or vestibular analyzer.

Attacks of vertigo (dizzy spells) are largely caused by changes in the functional interaction between the sympathetic and the parasympathetic nervous systems in the direction of predominance of function of parasympathetic system. These changes are accompanied by vasomotor disturbances in the internal ear with increase permeability of vascular walls and subsequent increase in the amount of endolymph in the vestibular apparatus. Vertigo is a typical sign of loss of vestibular apparatus of various origins, including dysfunction of vestibular nerve and vestibular cochlear system, disturbances of blood circulation in vertebral-basilar system, pathology of central nervous system (CNS) etc. Vertigo as manifestation of kinetosis, is accompanied by other vestibulo-vegetative disorders including three types of reactions: vestibule-motor (nystagmus and reactions of deviation), vestibular-sensory (except vertigo it can be nystagmus (or reaction of postrotation), protective movements) and vegetative (nausea, vomiting, hyperhidrosis, tachycardia, feeling of heat, vibration of pulse and blood pressure).

Known in the art is the homeopathic medication “AVIAMORE” (RU 2113230 C1, A61K 35/78, 1998) which is based on vegetable raw material that is designed for treatment and prophylaxis of motion sickness (kinetosis) in the form of in transport, sea and air sickness. The efficiency of this medication in most cases is not very high.

Also known are neurotropic drugs on the basis of antiserum to brain specific protein S-100 (RU 2156621 C1, A61K39/395, Sep. 27, 2000).

There is a continuing need for new drug products with desired therapeutic efficacy for treatment of vertigo of various genesis, kinetosis and vegetative-vascular dystonia.

The therapeutic effect of an extremely diluted form (or ultra-low form) of antibodies potentized by homeopathic technology (activated potentiated form) has been discovered by the inventor of the present patent application, Dr. Oleg I. Epshtein. U.S. Pat. No. 7,582,294 discloses a medicament for treating Benign Prostatic Hyperplasia or prostatitis by administration of a homeopathically activated form of antibodies to prostate specific antigen (PSA). U.S. Pat. No. 7,700,096 discloses a homeopathically potentized form of antibodies to endothelial NO-synthase.

The S-100 protein is a cytoplasmic acidic calcium binding protein found predominantly in the gray matter of the brain, primarily in glia and Schwann cells. The protein exists in several homo- or heterodimeric isoforms consisting of two immunologically distinct subunits, alpha and beta. The S-100 protein has been suggested for use as an aid in the diagnosis and assessment of brain lesions and neurological damage due to brain injury, as in stroke. Yardan et al., Usefulness of S100B Protein in Neurological Disorders, J Pak Med Assoc Vol. 61, No. 3, March 2011, which is incorporated herein by reference.

Ultra low doses of antibodies to S-100 protein have been shown to have anxiolytic, anti-asthenic, anti-aggressive, stress-protective, anti-hypoxic, anti-ischemic, neuroprotective and nootropic activity. See Castagne V. et al., Antibodies to S100 proteins have anxiolytic-like activity at ultra-low doses in the adult rat, J Pharm Pharmacol. 2008, 60(3):309-16; Epshtein O. I., Antibodies to calcium-binding S100B protein block the conditioning of long-term sensitization in the terrestrial snail, Pharmacol Biochem Behav., 2009, 94(1):37-42; Voronina T. A. et al., Chapter 8. Antibodies to S-100 protein in anxiety-depressive disorders in experimental and clinical conditions. In “Animal models in biological psychiatry”, Ed. Kalueff A. V. N-Y, “Nova Science Publishers, Inc.”, 2006, pp. 137-152, all of which are incorporated herein by reference.

Nitric oxide (NO) is a gaseous molecule that has been shown to acts in the signaling of different biological processes. Endothelium-derived NO is a key molecule in regulation of vascular tone and its association with vascular disease has long been recognized. NO inhibits many processes known to be involved in the formation of atherosclerotic plaque, including monocyte adhesion, platelet aggregation and vascular smooth muscle cell proliferation. Another important role of endothelial NO is the protection of the vascular wall from the oxidative stress induced by its own metabolic products and by the oxidation products of lipids and lipoproteins. Endothelial dysfunction occurs at very early stages of atherosclerosis. It is therefore possible that deficiency in local NO availability could be a final common pathway that accelerates atherogenesis in humans. In addition to its role in the vascular endothelium, NO availability has been shown to modulate metabolism of lipoproteins. Negative correlation has been reported between plasma concentrations of NO metabolic products and plasma total and Low Density Lipoprotein [LDL] cholesterol levels while High Density Lipoprotein [HDL] improves vascular function in hypercholesterolaemic subjects. The loss of NO has considerable effect on the development of the disease. Diabetes mellitus is associated with increased rates of morbidity and mortality caused primarily by the accelerated development of atherosclerotic disease. Moreover, reports show that diabetics have impaired lung functions. It has been proposed that insulin resistance leads to airway inflammation. Habib et al., Nitric Oxide Measurement From Blood To Lungs, Is There A Link? Pak J Physiol 2007; 3(1). Nitric oxide is synthesized by the endothelium from L-arginine by nitric oxide synthase (NO synthase). NO synthase occurs in different isoforms, including a constitutive form (cNOS) and an inducible form (iNOS). The constitutive form is present in normal endothelial cells, neurons and some other tissues.

### SUMMARY

In one aspect, the present invention provides a combination pharmaceutical composition comprising activated-potentiated form of an antibody to brain-specific protein S-100 and activated-potentiated form of an antibody to endothelial NO synthase.

In one variant, the present invention provides a combination pharmaceutical composition comprising activated-potentiated form of an antibody to brain-specific protein S-100 and activated-potentiated form of an antibody to endothelial NO synthase, wherein the antibody is to the entire protein S-100 or fragments thereof.

In one variant, the present invention provides a combination pharmaceutical composition comprising activated-potentiated form of an antibody to brain-specific protein S-100 and activated-potentiated form of an antibody to endothelial NO synthase, wherein the antibody is to the entire NO synthase or fragments thereof.

In one variant, the combination pharmaceutical composition of this aspect of the invention includes activated-potentiated form of an antibody to protein S-100 which is in the form of a mixture of (C12, C30, and C50) or (C12, C30 and C200) homeopathic dilutions impregnated onto a solid carrier. The activated-potentiated form of an antibody to NO synthase is in the form of mixture of (C12, C30, and C50) or (C12, C30 and C200) homeopathic dilutions may be subsequently impregnated onto the solid carrier.

In one variant, the combination pharmaceutical composition of this aspect of the invention includes activated-potentiated form of an antibody to NO synthase which is in the form of a mixture of (C12, C30, and C50) or (C12, C30 and C200) homeopathic dilutions impregnated onto a solid carrier. The activated-potentiated form of an antibody to protein S-100 is in the form of mixture of (C12, C30, and C50) or (C12, C30 and C200) homeopathic dilutions may be subsequently impregnated onto the solid carrier.

Preferably, the activated-potentiated form of an antibody to protein S-100 is a monoclonal, polyclonal or natural antibody, more preferably, a polyclonal antibody. In one variant of this aspect of the invention, the activated-potentiated form of an antibody to a protein S-100 is prepared by successive centesimal dilutions coupled with shaking of every dilution. Vertical shaking is specifically contemplated

Preferably, the activated-potentiated form of an antibody to NO synthase is a monoclonal, polyclonal or natural antibody, more preferably, a polyclonal antibody. In one variant of this aspect of the invention, the activated-potentiated form of an antibody to NO synthase is prepared by successive centesimal dilutions coupled with shaking of every dilution. Vertical shaking is specifically contemplated

In another aspect, the invention provides the method of treating vertigo of various genesis, kinetosis and vegetative-vascular dystonia comprising administration to a subject in need thereof of a combination pharmaceutical composition comprising activated-potentiated form of an antibody to brain-specific protein S-100 and activated-potentiated form of an antibody to endothelial NO synthase.

In one variant the method of treatment administration to a subject in need thereof a combination pharmaceutical composition comprising activated-potentiated form of an antibody to brain-specific protein S-100 and activated-potentiated form of an antibody to endothelial NO synthase wherein said administration of said combination leads to a significant improvement in motion sickness as measured by tolerance of CCEAC test.

In one variant the method of treatment administration to a subject in need thereof a combination pharmaceutical composition comprising activated-potentiated form of an antibody to brain-specific protein S-100 and activated-potentiated form of an antibody to endothelial NO synthase wherein said administration of said combination leads to a significant improvement in the stabilizing effect on the balance of autonomic nervous system as measured by CCEAC test.

In one variant of the invention, there is provided administration of from one to two unit dosage forms of the activated-potentiated form of an antibody to protein S-100 and one to two unit dosage forms of the activated-potentiated form of an antibody to NO synthase, each of the dosage form being administered from once daily to four times daily. Preferably, the one to two unit dosage forms of each of the activated-potentiated forms of an antibody is administered twice daily.

### DETAILED DESCRIPTION

The invention is defined with reference to the appended claims. With respect to the claims, the glossary that follows provides the relevant definitions.

The term “antibody” as used herein shall mean an immunoglobulin that specifically binds to, and is thereby defined as complementary with, a particular spatial and polar organization of another molecule. Antibodies as recited in the claims may include a complete immunoglobulin or fragment thereof, may be natural, polyclonal or monoclonal, and may include various classes and isotypes, such as IgA, IgD, IgE, IgG1, IgG2a, IgG2b and IgG3, IgM, etc. Fragments thereof may include Fab, Fv and F(ab′)₂, Fab′, and the like. The singular “antibody” includes plural “antibodies”.

The term “activated-potentiated form” or “potentiated form” respectively, with respect to antibodies recited herein is used to denote a product of homeopathic potentization of any initial solution of antibodies. “Homeopathic potentization” denotes the use of methods of homeopathy to impart homeopathic potency to an initial solution of relevant substance. Although not so limited, “homeopathic potentization” may involve, for example, repeated consecutive dilutions combined with external treatment, particularly vertical (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰⁰ and 100²⁰⁰ times, respectively, which is equivalent to centesimal homeopathic dilutions (C12, C30, and C200) or the use of the mixture of three aqueous or aqueous-alcohol dilutions of the primary matrix solution of antibodies diluted 100¹², 100³⁰ and 100⁵⁰ times, respectively, which is equivalent to centesimal homeopathic dilutions (C12, C30 and C50). Examples of homeopathic potentization are described in U.S. Pat. Nos. 7,572,441 and 7,582,294, which are incorporated herein by reference in their entirety and for the purpose stated. While the term “activated-potentiated form” is used in the claims, the term “ultra-low doses” is used in the examples. The term “ultra-low doses” became a term of art in the field of art created by study and use of homeopathically diluted and potentized form of substance. The term “ultra-low dose” or “ultra-low doses” is meant as fully supportive and primarily synonymous with the term “activated-potentiated” form used in the claims.

In other words, an antibody is in the “activated-potentiated” or “potentiated” form when three factors are present. First, the “activated-potentiated” form of the antibody is a product of a preparation process well accepted in the homeopathic art. Second, the “activated-potentiated” form of antibody must have biological activity determined by methods well accepted in modern pharmacology. And third, the biological activity exhibited by the “activated potentiated” form of the antibody cannot be explained by the presence of the molecular form of the antibody in the final product of the homeopathic process.

For example, the activated potentiated form of antibodies may be prepared by subjecting an initial, isolated antibody in a molecular form to consecutive multiple dilutions coupled with an external impact, such as mechanical shaking. The external treatment in the course of concentration reduction may also be accomplished, for example, by exposure to ultrasonic, electromagnetic, or other physical factors. V. Schwabe “Homeopathic medicines”, M., 1967, U.S. Pat. Nos. 7,229,648 and 4,311,897, which are incorporated by reference in their entirety and for the purpose stated, describe such processes that are well accepted methods of homeopathic potentiation in the homeopathic art. This procedure gives rise to a uniform decrease in molecular concentration of the initial molecular form of the antibody. This procedure is repeated until the desired homeopathic potency is obtained. For the individual antibody, the required homeopathic potency can be determined by subjecting the intermediate dilutions to biological testing in the desired pharmacological model. Although not so limited, “homeopathic potentization” may involve, for example, repeated consecutive dilutions combined with external treatment, particularly (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably, water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions C12, C30 and C200 or the mixture of three aqueous or aqueous-alcohol dilutions of the primary matrix solution (mother tincture) of antibodies diluted 100¹², 100³⁰ and 100⁵⁰ times, respectively, which is equivalent to centesimal homeopathic dilutions C12, C30 and C50. Examples of how to obtain the desired potency are also provided, for example, in U.S. Pat. Nos. 7,229,648 and 4,311,897, which are incorporated by reference for the purpose stated. The procedure applicable to the “activated potentiated” form of the antibodies described herein is described in more detail below.

There has been a considerable amount of controversy regarding homeopathic treatment of human subjects. While the present invention relies on accepted homeopathic processes to obtain the “activated-potentiated” form of antibodies, it does not rely solely on homeopathy in human subjects for evidence of activity. It has been surprisingly discovered by the inventor of the present application and amply demonstrated in the accepted pharmacological models that the solvent ultimately obtained from consecutive multiple dilution of a starting molecular form of an antibody has definitive activity unrelated to the presence of the traces of the molecular form of the antibody in the target dilution. The “activated-potentiated” form of the antibody provided herein are tested for biological activity in well accepted pharmacological models of activity, either in appropriate in vitro experiments, or in vivo in suitable animal models. The experiments provided further below provide evidence of biological activity in such models. Human clinical studies also provide evidence that the activity observed in the animal model is well translated to human therapy. Human studies have also provided evidence of availability of the “activated potentiated” forms described herein to treat specified human diseases or disorders well accepted as pathological conditions in the medical science.

Also, the claimed “activated-potentiated” form of antibody encompasses only solutions or solid preparations the biological activity of which cannot be explained by the presence of the molecular form of the antibody remaining from the initial, starting solution. In other words, while it is contemplated that the “activated-potentiated” form of the antibody may contain traces of the initial molecular form of the antibody, one skilled in the art could not attribute the observed biological activity in the accepted pharmacological models to the remaining molecular form of the antibody with any degree of plausibility due to the extremely low concentrations of the molecular form of the antibody remaining after the consecutive dilutions. While the invention is not limited by any specific theory, the biological activity of the “activated-potentiated” form of the antibodies of the present invention is not attributable to the initial molecular form of the antibody. Preferred is the “activated-potentiated” form of antibody in liquid or solid form in which the concentration of the initial molecular form of the antibody is below the limit of detection of the accepted analytical techniques, such as capillary electrophoresis and High Performance Liquid Chromatography. Particularly preferred is the “activated-potentiated” form of antibody in liquid or solid form in which the concentration of the initial molecular form of the antibody is below the Avogadro number. In the pharmacology of molecular forms of therapeutic substances, it is common practice to create a dose-response curve in which the level of pharmacological response is plotted against the concentration of the active drug administered to the subject or tested in vitro. The minimal level of the drug which produces any detectable response is known as a threshold dose. It is specifically contemplated and preferred that the “activated-potentiated” form of the antibodies contains molecular antibody, if any, at a concentration below the threshold dose for the molecular form of the antibody in the given biological model.

Test used in the present application are described below.

(1) Test with continuous cumulative effect of accelerations by Coriolis (CCEAC) refers to a test that can detect the stability of a subject to Coriolis effect of accelerations and thus may indicate the degree of sensitivity of a subject to motion sickness. (Markaryan et al., Vestibular selection by the method of continuous cumulative effect of accelerations by Coriolis, Military medical magazine, 1966. No. 9. Pages 59-62; Voyenizdat, Research Methodologies In Medical And Flight Inspection, 1972).

The order of test performance is as follows: The subject is sited in a Barany rotation chair or in an electrorotation chair in a position such that the axis of rotation is along the body. Eyes are closed. With the constant rotation of the chair at the rate of 180 deg/sec. (one turn per two seconds) the subjects at the end of fifth turn, are instructed to tilt their head from right shoulder to the left shoulder or from the left shoulder to the right shoulder and back at an angle of not less than 30 degrees in each direction from the vertical. The flexions are carried out continuously without excessive tension of the neck muscles and turns of a head during all rotation period. Thus, every movement of the head from shoulder to shoulder runs smoothly for 2 seconds without stopping in the middle or at peak positions. Tilt speed is controlled by a metronome or time pronouncing numbers 21 and 22 which should correspond to 2 seconds. The time necessary to run test starting from the first jactatio capitis.

Before the test the subject is instructed to report any appearance of the illusion of swing, feeling of heat, fever, salivation, nausea which may occur during the test. Before the test, the subject is instructed to perform a few test head movements so that the subject is comfortable with speed control of oscillating motions and is able to adopt the correct position of head at the time of movement.

The appearance of marked vestibular vegetative disorders (pallor, hyperhidrosis, nausea, retching) during the continuous performance of CCEAC test is the criteria of limit tolerance of effects of Coriolis accelerations. The time of occurrence of vestibular-autonomic responses is registered from the start of the CCEAC test and the time of its termination after completion of the CCEAC test performance. Tests on the tolerance of Coriolis accelerations were carried out in the first half of a day not earlier than 2 hours after meals and only once a day. On the day of test the subject was not longer exposed to other influences (in the altitude chamber, centrifuge, etc.).

(2) The methodology of quantitative evaluation of disorders of vestibular-vegetative sensibility (Halle's scale) is based on an assessment of evidence (in points) of the vestibular-vegetative symptoms (dizziness, nausea, sweating, skin pallor, drowsiness, etc.) occurring during the CCEAC test performance. The technique enables the identification of the degree of human tolerance of Coriolis accelerations (poor, satisfactory, good and excellent). (Quantitative evaluation of disorders of vestibular-vegetative sensibility, Cosmic biology and aeroastromedicine, 1981, No. 3, pages 72-75).

(3) Study of heart rate variability (HRV) is used to collect data on HRV the Biocom Wellness Scan system. It was developed by AWS, LLC., and created in accordance with International Standard of European Cardiologists Association and North American Electrophysiology Association (International Task Force consisting of the European Society of Cardiology and the North American Society for Pacing and Electrophysiology, 1996). (Task Force of the European Society of Cardiology and the North American Society of Pacing and Electrophysiology. Heart Rate Variability standards of measurement, physiological interpretation, and clinical use. Cir. 1996; 93:1043 1065).

The following equipments are used:

1. Personal computer (PC) with operating system Windows.

2. Photoplethysmograph HRM-02 (PPG).

3. Ear sensor (PPG ear-clip).

4. Software Biocom Wellness Scan Software on CD.

5. Instruction for use in electronic format (PDF).

The subject undergoes three tests of autonomic balance assessment: 5-minutes record of HRV at rest; breathing test; orthostatic test.

Procedure of HRV Study

1. Prior to start of the test, the researcher gives to the subject a short description of each test.

2. The subject sits in a comfortable and relaxed position.

3. Ear sensor is wiped with an alcoholic solution and placed on the ear lobe. Earrings, if any, must be removed before the test.

4. The researcher records 5-minutes of HRV at rest (Short-term Resting HRV Test) for performance.

5. The researcher administers the test according to the guidelines.

6. Straight after the test is finished and data is recorded in a database, the researcher selects the next test which is either breathing (Metronome Breathing Test) or an orthostatic test.

7. The researcher follows the guidelines to administer the breathing or orthostatic tests.

8. Immediately after the test is finished and data is recorded in the database and the researcher reviews the results of all performed tests to determine if the test was properly administered.

9. At the end of data review the test is terminated and the ear sensor is removed from the subject's ear.

Procedure for the 5-Minutes Record of HRV at Rest

Short-term HRV test is used to evaluate the balance between sympathetic and parasympathetic branches of the autonomic nervous system. It is a 5-minute record of photoplethysmography performed in a sitting position without provocative maneuvers. During test the study participant is instructed to breath at random with respiratory rate of at least 9 breaths per minute to obtain valid parameters of HRV. The next HRV parameters are calculated:

1. Parameters in time area are as follows:
(a) HR which is the mean value of heart rate, measured in meats/per/minute (BPM).
(b) Mean NN which is mean value of inter-bit interval, measured in milliseconds.
(c) SDNN which is the standard deviation of NN intervals. Since the quantity under the square root is mathematically equivalent to the total power in spectral analysis the SDNN reflects all cyclic components responsible for variability. The actual value of SDNN depend on the length of record—the longer the record, the higher the SDNN value. Thus, in practice it is impossible to compare the values of SDNN calculated at different time intervals. SDNN is measured in milliseconds.
(d) RMS-SD which is the square root of the differences between successive NN intervals. This indicator assesses the high-frequency component of heart rate variability which is associated with the parasympathetic regulation of a heart. RMS-SD is measured in milliseconds.

All parameters of HRV in time area are calculated on the normal inter-bit intervals (NN) due to normal sinus heartbeat recorded during the test.

2. Parameters in frequency area are as follows:
(a) Total Power (TP) is the assessment of power spectrum density in the range from 0 to 0.4 Hz. This indicator reflects the overall activity of the autonomic nervous system, at that sympathetic activity contributes the most investment. Total Power is calculated in milliseconds squared (ms²).
(b) Very Low Frequency (VLF) is a power spectrum density in the range between 0.0033 and 0.04 Hz. The physiological nature of this index is that it is an indicator of the total activity of various slow mechanisms of regulation. VLF is calculated in milliseconds squared (ms²).
(c) Low Frequency (LF) is a power spectrum density in the range between 0.04 and 0.15 Hz. This figure reflects both sympathetic and parasympathetic activity. It is a good indicator of sympathetic activity in long-term records of HRV. Parasympathetic influence is represented in LF when the respiratory rate is less than 9 breaths per minute. LF is calculated in square milliseconds (ms²).
(d) High Frequency (HF) is a power spectrum density in the range between 0.15 and 0.4 Hz. This indicator reflects the parasympathetic activity. HF is also known as “respiratory” component since it corresponds to variations of NN intervals caused by breathing (a phenomenon known as respiratory sinus arrhythmia (RSA)). The heart rate increases during breath in and decreases during exhalation. HF is calculated in square milliseconds (ms²).
(e) LF/HF Ratio is the ratio between the density of the power spectrum in the range of LF and HF. This indicator reflects the overall balance between sympathetic and parasympathetic activity. High values of this index are indicators of dominance of sympathetic activity while the lowest—the parasympathetic one. LF/HF Ratio is calculated in normalized units.
(f) Normalized Low Frequency (LF norm) is the ratio between the absolute value of the LF and TP without VLF. This index minimizes the effect of VLF influence in the overall power spectrum and highlights the changes in sympathetic regulation. HLF norm is calculated in percents.
(g) Normalized High Frequency (HF norm) is the ratio between the absolute value of the HF and TP without VLF. This index minimizes the effect of VLF influence in the overall power spectrum and highlights the changes in parasympathetic regulation. HFnorm is calculated in percents.
Frequency HRV parameters are calculated from the of the power spectrum density (PSD) calculated by the fast Fourier transformation (FFT).

(5) Description of breathing test. This test is designed to assess the parasympathetic branch of the autonomic nervous system. The test gives a positive stimulation of the parasympathetic regulation of heart rhythm.

During this test the subject is instructed to breathe deeply and evenly with respiratory rate of 6 breaths per minute. During the test it is important to exclude any events that may affect random breathing such as talking, coughing, sighing, etc. This interference can cause unwanted fluctuations in heart rate and can distort the results. The subject was instructed to breathe for 1 minute following the movement of an object being shown on the screen. The following test parameters are calculated:

1. Minimal HR (bpm);

2. Maximal HR (bpm);

3. Standard Deviation of HR (bpm);

4. Mean ratio of HR max/HR min (E/I Ratio); and

5. Maximal Variance of HR during test (bpm).

(6) Description of orthostatic test. This test is used to evaluate the effect of parasympathetic regulation of the rhythm of the heart. The test is based on changes in the position of the body of the subject. The subject must be relaxed in a sitting position. After recording of the heart rhythm for a minute, the subject is instructed to stand up avoiding any sudden movements. The subject remains standing for one more minute. Monitoring of heart rhythm continues throughout the test. The purpose of recording the base line and maneuver of standing up is to evaluate the unsteady transition process in the rhythm of the heart caused by a change in body position. Heart rate is monitored until the heart rate stabilizes. The following test parameters are calculated:

1. 30:15 Ratio (which is the ratio between the maximum heart rate value during the first 15 seconds after standing up to the minimum value of heart rate during the first 30 seconds after standing up or exercise reaction, c.u.).

2. The time of gain the maximal HR value after recovery (or reaction time, sec.).

3. The time of gain HR 75% of level of base line (or stabilization time, sec.).

4. Minimal HR value (b/p/s).

5. Maximal HR value (b/p/s).

(7) The self-esteem of functional state (WBAM). This test permits the numerical characterization of three types of subjective states: well-being, activity and mood (WBAM) which are determined by using a special formsheet. In the formsheet there are 30 pairs of words of opposite meaning and between there is rating scale. Depending on the subjective assessment of self condition the subject notes the evidence degree of one or another features on a seven-point scale. Signs of the numbers describe: 1-2, 7-8, 13-14, 19-20, 25-26—well-being, 3-4, 9-10, 15-16, 21-22, 27-28—activity, 5-6, 11-12, 17-18, 23-24, 29-30—mood. When processing the results of well-being and mood the assessments are re-coded from 7 to 9 from left to right and activity—from right to left. (Doskin, et al., The Test Of differentiate Self-esteem Of Functional State, Psychological questions, 1973, No. 6, pages 141-145).

For each feature (well-being, activity, mood) the mean arithmetic value is calculated, its error and standard deviation. It gives the possibility to integrally assess the subjective state. The mean arithmetic value is a direct subjective characteristic of the functional state and performance capability and by the dispersion volume of assessments within one group of features (standard deviation) it can be judged about the validity of found results.

(8) Psychometric tests. This test is performed using a computer program “OKO” (operational control of the operator) developed “Livability and health care of personnel of Navy,” for Central Research Institute of Shipbuilding for Russian Defense Ministry, led by Professor V. Yu. Rybnikov.

The following psycho physiological parameters are determined:
Reaction on moving object (RMO);
Simple motor reaction time (SMRT);
Range of attention (RA); and
Attention span (AS).

Due to the high variability of psychophysiological indicators, measurements are performed several times and then the mean arithmetic value of the entire series is calculated. In particular, the SMRT assessment was repeated 50 times, RMO-20 times, RA and AS-5 times. They also calculated in RMO test of 20 values the number of hits on target and then calculated the percentage of accurate hits. In AS test they studied the average time of the test performance, the number of correct answers in percents to its total number executed by the subjects.

To integrate the indicators they measured attention stability factor (ASF) which was calculated by dividing of percent of correct answers on average time of test performance.

(9) The reaction to a moving object (RMO). The reaction to a moving object allows for the determination of the accuracy of a subject's response to a stimulus and evaluation as to the balance of excitation and inhibition processes in the cerebral cortex. The essence of the reaction is necessary to stop the rapid movement of an object in a pre-fixed point. For this an electro-stopwatch may be applied switched on with remote control by the researcher, the second hand of which the subject has to stop exactly on the mark “0” by pressing the button on his remote control. This test can also be performed using a special computer program on a PC. The response of the subject may be immature—the hand of electro-stopwatch did not reach the “0” mark, delayed—the hand jumped over the “0” mark, accurate—the hand stopped on the mark “0”. Each immature or delayed reaction has quantitative characteristics in absolute units. To assess the results of tests performed the relative accuracy of answers is calculated (in % of total responses) as well as mean arithmetic and mean algebraic values of deviations of all shown reactions. (Zheglov, et al., The Retention Of Performance Capability Of Sailing Personnel Of Navy. Guidance For Doctors, 1990, page 192).

(10) Simple sensomotor reaction on light signal or simple motor reaction time (SMRT). Simple motor reaction time is a technique to characterize the strength of the nervous processes. In a simple sensomotor reaction two mental acts can be distinguished: the percipiency (sensory moment of reaction) and the response move (motor component). The SMRT assessment can be made in the traditional way (using chronoreflexometers) as well as the use of special computer programs. Prior to testing, the researcher explains the rules of the test to the subject. Then the subject is instructed to sit on a chair, to put his hands on the table before chronoreflexometer and put the finger of the leading hand in its corresponding button. When the subject is ready the physician-researcher gives the command and after 3-10 seconds switches on the device. The task of the subject is to respond as soon as possible after the onset of the signal by pressing a button and turn off the light bulb. The simple motor reaction time is measured (in milliseconds) since the moment of occurrence of the special object on the monitor screen before pressing the button by the subject on manipulator (keyboard or mouse). SMRT is measured typically for 50 times after which the arithmetic mean value of the indicator is determined. (Zheglov, et al., The Retention Of Performance Capability Of Sailing Personnel Of Navy. Guidance For Doctors, 1990, page 192).

(11) Harvard step-test. This is a functional test which allows for the identification of the reaction of the cardiovascular system to adverse effects and in particular the impact of Coriolis acceleration, the 2-minute Harvard step-test was used (V. L. Karpman, et.al., 1988; Novicov, et al, Study methods in physiology of military labour. Guidance, 1993, page 240).

The technique is based on an assessment of autonomic shifts in the performance of squats and recovery possibilities of a body to normalize the heart rate.

The value of step-test characterizes the rate of recovery processes after intense enough muscular work. The faster the pulse restores, the lower the value of (P2+P3+P4) and therefore, the higher step test index.

In athletes this index is usually higher than non-athletes. The index is expected to be reduced in subjects with drug toxicity. At the same time, increases in the index indicate that the drug increases the functional reserves of a body and the ability to tolerate adverse environmental impacts, including kinetic actions.

The test is performed with the subject squatting for 2 minutes at the rate of 30 times per minute. On the 2nd, 3rd and 4th minutes after squatting the pulse is measured on for the first 30 seconds of every minute. The step-test index was calculated using the formula:

Harvard step-test index=T*100/(P2+P3+P4)*2,
where T is squatting time in sec.; P2, P3, P4 is pulse frequency on 2-nd, 3-rd and 4-th minutes of recovery period, *—multiple sign.

Due to the fact that drugs are allocated to persons amenable to motion sickness including drivers, its safety was assessed in carrying out responsible operator functions by persons. In order to determine the key predictors for quality of activity of operational types, a detailed study of the functional state of the central nervous system (such as state of systems of coordination and response, systems which provide high efficiency of fine motor components of activity as well as systems of attention) was performed.

(12) Stange's test. The essence of the Stange's test is to hold the breath after three breaths in for ¾ of full depth of inhalation. Prior to the test the nose of the subject was clipped or the subject pressed his nose with his fingers. The length of time that the subject held its breath was recorded by stopwatch. (Zheglov, et al, The Retention Of Performance Capability Of Sailing Personnel Of Navy. Guidance For Doctors, 1990, page 192).

The test may be carried out twice at intervals of 3-5 minutes between determinations. The test is assessed by the duration of the breath as follows:
Less than 39 sec.—unsatisfactory;
40-9 sec.—satisfactory;
More than 50 sec.—good.

(13) Gench's test. The essence of the test performance is to hold the breath at exhalation after three breaths. (Zheglov, et al., The Retention Of Performance Capability Of Sailing Personnel Of Navy. Guidance For Doctors, 1990, page 192). When conducting the Gench's test in prone position the duration of breath holding in healthy subjects is 25-30 seconds. When it is repeated after the walking stage (44 m in 30 sec.) the duration of breath holding is reduced to 17-22 seconds and with a functional deficiency of a body, it is reduced up to 5-15 seconds. Assessment of the test was carried out as follows:
Less than 34 sec.—unsatisfactory;
35-39 sec.—satisfactory;
More than 40 sec.—good.

In one aspect, the present invention provides a combination pharmaceutical composition comprising a) an activated-potentiated form of an antibody to NO synthase and b) an activated-potentiated form of an antibody to brain-specific protein S-100. As set forth herein above, each of the individual components of the combination is generally known for its won individual medical uses. However, the inventors of the present application surprisingly discovered that administration of the combination remarkably is useful for the treatment of vertigo of various genesis, kinetosis and vegetative-vascular dystonia.

In another aspect, the invention provides the method of treatment of vegetative-vascular dystonia and symptoms thereof by means of insertion in an organism of activated-potentiated form of antibodies to brain-specific protein S-100 simultaneously with activated-potentiated form of antibodies to endothelial NO synthase in ultra-low doses of affinity purified antibodies.

Preferably, for the purpose of treatment, the combination pharmaceutical composition is administered from once daily to four times daily, each administration including one or two combination unit dosage forms.

The pharmaceutical composition of the present application for the purpose of treatment of vertigo of various genesis, kinetosis and vegetative-vascular dystonia contains active components in volume primarily in 1:1 ratio.

For the purpose of treatment of vertigo of various genesis, kinetosis and vegetative-vascular dystonia the components of the pharmaceutical composition may be administered separately. However, the simultaneous administration of the combined components in one form of solutions and/or solid dosage form (tablet), which contains activated-potentiated form of antibodies to brain-specific protein S-100 and, accordingly, activated-potentiated form of antibodies to endothelial NO synthase is preferred.

In addition, during treatment of vertigo of various genesis, kinetosis and vegetative-vascular dystonia, separate and simultaneous application (intake to organism) of the declared pharmaceutical composition in the form of two separately prepared medications both in the form of solutions and solid dosage forms (tablets) each of which contains activated-potentiated form of antibodies to endothelial NO-synthase or to S-100 protein is possible.

The medical product is prepared mainly as follows.

The combination pharmaceutical composition in accordance with the present invention may be in the liquid form or in solid form. Each of the activated potentiated forms of the antibodies included in the pharmaceutical composition is prepared from an initial molecular form of the antibody via a process accepted in homeopathic art. The starting antibodies may be monoclonal, or polyclonal antibodies prepared in accordance with known processes, for example, as described in Immunotechniques, G. Frimel, M., “Meditsyna”, 1987, p. 9-33; “Hum. Antibodies. Monoclonal and recombinant antibodies, 30 years after” by Laffly E., Sodoyer R.—2005—Vol. 14.—N1-2. P.33-55, both incorporated herein by reference.

Monoclonal antibodies may be obtained, e.g., by means of hybridoma technology. The initial stage of the process includes immunization based on the principles already developed in course of polyclonal antisera preparation. Further stages of work involve production of hybrid cells generating clones of antibodies with identical specificity. Their separate isolation is performed using the same methods as in case of polyclonal antisera preparation.

Polyclonal antibodies may be obtained via active immunization of animals. For this purpose, for example, suitable animals (e.g. rabbits) receive a series of injections of the appropriate antigen: brain-specific protein S-100 and endothelial NO synthase. The animals' immune system generates corresponding antibodies, which are collected from the animals in a known manner. This procedure enables preparation of a monospecific antibody-rich serum.

If desired, the serum containing antibodies may be purified, e.g., using affine chromatography, fractionation by salt precipitation, or ion-exchange chromatography. The resulting purified, antibody-enriched serum may be used as a starting material for preparation of the activated-potentiated form of the antibodies. The preferred concentration of the resulting initial solution of antibody in the solvent, preferably, water or water-ethyl alcohol mixture, ranges from about 0.5 to about 5.0 mg/ml.

The preferred procedure for preparing each component is the use of the mixture of three aqueous-alcohol dilutions of the primary matrix solution of antibodies diluted 100¹², 100³⁰ and 100²⁰⁰ times, respectively, which is equivalent to centesimal homeopathic dilutions C12, C30 and C200. To prepare a solid dosage form, a solid carrier is treated with the desired dilution obtained via the homeopathic process. To obtain a solid unit dosage form of the combination of the invention, the carrier mass is impregnated with each of the dilutions. Both orders of impregnation are suitable to prepare the desired combination dosage form.

In a preferred embodiment, the starting material for the preparation of the activated potentiated form that comprise the combination of the invention is polyclonal antibodies to brain-specific protein S-100 and endothelial NO synthase an initial (matrix) solution with concentration of 0.5 to 5.0 mg/ml is used for the subsequent preparation of activated-potentiated forms.

To prepare the pharmaceutical composition preferably polyclonal antibodies to brain-specific protein S-100 and endothelial NO synthase are used.

Polyclonal antibodies to endothelial NO synthase are obtained using adjuvant as immunogen (antigen) for immunization of rabbits and whole molecule of bovine endothelial NO synthase of the following sequence:

```
SEQ.ID. NO. 1
Met Gly Asn Leu Lys Ser Val Gly Gln Glu Pro Gly Pro Pro Cys
  1 5 10 15
Gly Leu Gly Leu Gly Leu Gly Leu Gly Leu Cys Gly Lys Gln Gly
  16 20 25 30
Pro Ala Ser Pro Ala Pro Glu Pro Ser Arg Ala Pro Ala Pro Ala
  31 35 40 45
Thr Pro His Ala Pro Asp His Ser Pro Ala Pro Asn Ser Pro Thr
  46 50 55 60
Leu Thr Arg Pro Pro Glu Gly Pro Lys Phe Pro Arg Val Lys Asn
  61 65 70 75
Trp Glu Leu GLys er Ile Thr Tyr Asp Thr Leu Cys Ala Gln Ser
  76 80 85 90
Gln Gln Asp Gly Pro Cys Thr Pro Arg Cys Cys Leu GLys er Leu
  91 95 100 105
Val Leu Pro Arg Lys Leu Gln Thr Arg Pro Ser Pro Gly Pro Pro
106 110 115 120
Pro Ala Glu Gln Leu Leu Ser Gln Ala Arg Asp Phe Ile Asn Gln
121 125 130 135
Tyr Tyr Ser Ser Ile Lys Arg Ser GLys er Gln Ala His Glu Glu
136 140 145 150
Arg Leu Gln Glu Val Glu Ala Glu Val Ala Ser Thr Gly Thr Tyr
151 155 160 165
His Leu Arg Glu Ser Glu Leu Val Phe Gly Ala Lys Gln Ala Trp
166 170 175 180
Arg Asn Ala Pro Arg Cys Val Gly Arg Ile Gln Trp Gly Lys Leu
181 185 190 195
Gln Val Phe Asp Ala Arg Asp Cys Ser Ser Ala Gln Glu Met Phe
196 200 205 210
Thr Tyr Ile Cys Asn His Ile Lys Tyr Ala Thr Asn Arg Gly Asn
211 215 220 225
Leu Arg Ser Ala Ile Thr Val Phe Pro Gln Arg Ala Pro Gly Arg
226 230 235 240
Gly Asp Phe Arg Ile Trp Asn Ser Gln Leu Val Arg Tyr Ala Gly
241 245 250 255
Tyr Arg Gln Gln Asp GLys er Val Arg Gly Asp Pro Ala Asn Val
256 260 265 270
Glu Ile Thr Glu Leu Cys Ile Gln His Gly Trp Thr Pro Gly Asn
271 275 280 285
Gly Arg Phe Asp Val Leu Pro Leu Leu Leu Gln Ala Pro Asp Glu
286 290 295 300
Ala Pro Glu Leu Phe Val Leu Pro Pro Glu Leu Val Leu Glu Val
301 305 310 315
Pro Leu Glu His Pro Thr Leu Glu Trp Phe Ala Ala Leu Gly Leu
316 320 325 330
Arg Trp Tyr Ala Leu Pro Ala Val Ser Asn Met Leu Leu Glu Ile
331 335 340 345
Gly Gly Leu Glu Phe Ser Ala Ala Pro Phe Ser Gly Trp Tyr Met
346 350 355 360
Ser Thr Glu Ile Gly Thr Arg Asn Leu Cys Asp Pro His Arg Tyr
361 365 370 375
Asn Ile Leu Glu Asp Val Ala Val Cys Met Asp Leu Asp Thr Arg
376 380 385 390
Thr Thr Ser Ser Leu Trp Lys Asp Lys Ala Ala Val Glu Ile Asn
391 395 400 405
Leu Ala Val Leu His Ser Phe Gln Leu Ala Lys Val Thr Ile Val
406 410 415 420
Asp His His Ala Ala Thr Val Ser Phe Met Lys His Leu Asp Asn
421 425 430 435
Glu Gln Lys Ala Arg Gly Gly Cys Pro Ala Asp Trp Ala Trp Ile
436 440 445 450
Val Pro Pro Ile Ser GLys er Leu Thr Pro Val Phe His Gln Glu
451 455 460 465
Met Val Asn Tyr Ile Leu Ser Pro Ala Phe Arg Tyr Gln Pro Asp
466 470 475 480
Pro Trp Lys GLy Ser Ala Thr Lys Gly Ala Gly Ile Thr Arg Lys
481 485 490 495
Lys Thr Phe Lys Glu Val Ala Asn Ala Val Lys Ile Ser Ala Ser
496 500 505 510
Leu Met Gly Thr Leu Met Ala Lys Arg Val Lys Ala Thr Ile Leu
511 515 510 525
Tyr Ala Ser Glu Thr Gly Arg Ala Gln Ser Tyr Ala Gln Gln Leu
526 530 535 540
Gly Arg Leu Phe Arg Lys Ala Phe Asp Pro Arg Val Leu Cys Met
541 545 550 555
Asp Glu Tyr Asp Val Val Ser Leu Glu His Glu Ala Leu Val Leu
556 560 565 570
Val Val Thr Ser Thr Phe Gly Asn Gly Asp Pro Pro Glu Asn Gly
571 575 580 585
Glu Ser Phe Ala Ala Ala Leu Met Glu Met Ser Gly Pro Tyr Asn
586 590 595 600
Ser Ser Pro Arg Pro Glu Gln His Lys Ser Tyr Lys Ile Arg Phe
601 605 610 615
Asn Ser Val Ser Cys Ser Asp Pro Leu Val Ser Ser Trp Arg Arg
616 620 625 630
Lys Arg Lys Glu Ser Ser Asn Thr Asp Ser Ala Gly Ala Leu Gly
631 635 640 645
Thr Leu Arg Phe Cys Val Phe Gly Leu GLy Ser Arg Ala Tyr Pro
646 650 655 660
His Phe Cys Ala Phe Ala Arg Ala Val Asp Thr Arg Leu Glu Glu
661 665 670 675
Leu Gly Gly Glu Arg Leu Leu Gln Leu Gly Gln Gly Asp Glu Leu
676 680 685 690
Cys Gly Gln Glu Glu Ala Phe Arg Gly Trp Ala Lys Ala Ala Phe
691 695 700 705
Gln Ala Ser Cys Glu Thr Phe Cys Val Gly Glu Glu Ala Lys Ala
706 710 715 720
Ala Ala Gln Asp Ile Phe Ser Pro Lys Arg Ser Trp Lys Arg Gln
721 725 730 735
Arg Tyr Arg Leu Ser Thr Gln Ala Glu Gly Leu Gln Leu Leu Pro
736 740 745 750
Gly Leu Ile His Val His Arg Arg Lys Met Phe Gln Ala Thr Val
751 755 760 765
Leu Ser Val Glu Asn Leu Gln Ser Ser Lys Ser Thr Arg Ala Thr
766 770 775 780
Ile Leu Val Arg Leu Asp Thr Ala Gly Gln Glu Gly Leu Gln Tyr
781 785 790 795
Gln Pro Gly Asp His Ile Gly Ile Cys Pro Pro Asn Arg Pro Gly
796 800 805 810
Leu Val Glu Ala Leu Leu Ser Arg Val Glu Asp Pro Pro Pro Pro
811 815 820 825
Thr Glu Ser Val Ala Val Glu Gln Leu Glu Lys GLys er Pro Gly
826 830 835 840
Gly Pro Pro Pro Ser Trp Val Arg Asp Pro Arg Leu Pro Pro Cys
841 845 850 855
Thr Leu Arg Gln Ala Leu Thr Phe Phe Leu Asp Ile Thr Ser Pro
856 860 865 870
Pro Ser Pro Arg Leu Leu Arg Leu Leu Ser Thr Leu Ala Glu Glu
871 875 880 885
Pro Ser Glu Gln Gln Glu Leu Glu Thr Leu Ser Gln Asp Pro Arg
886 890 895 900
Arg Tyr Glu Glu Trp Lys Trp Phe Arg Cys Pro Thr Leu Leu Glu
901 905 910 915
Val Leu Glu Gln Phe Pro Ser Val Ala Leu Pro Ala Pro Leu Leu
916 920 925 930
Leu Thr Gln Leu Pro Leu Leu Gln Pro Arg Tyr Tyr Ser Val Ser
931 935 940 945
Ser Ala Pro Asn Ala His Pro Gly Glu Val His Leu Thr Val Ala
946 950 955 960
Val Leu Ala Tyr Arg Thr Gln Asp Gly Leu Gly Pro Leu His Tyr
961 965 970 975
Gly Val Cys Ser Thr Trp Leu Ser Gln Leu Lys Thr Gly Asp Pro
976 980 985 990
Val Pro Cys Phe Ile Arg Gly Ala Pro Ser Phe Arg Leu Pro Pro
991 995 1000 1005
Asp Pro Tyr Val Pro Cys Ile Leu Val Gly Pro Gly Thr Gly Ile
1006 1010 1015 1020
Ala Pro Phe Arg Gly Phe Trp Gln Glu Arg Leu His Asp Ile Glu
1021 1025 1030 1035
Ser Lys Gly Leu Gln Pro Ala Pro Met Thr Leu Val Phe Gly Cys
1036 1140 1145 1050
Arg Cys Ser Gln Leu Asp His Leu Tyr Arg Asp Glu Val Gln Asp
1051 1155 1160 1065
Ala Gln Glu Arg Gly Val Phe Gly Arg Val Leu Thr Ala Phe Ser
1066 1170 1175 1080
Arg Glu Pro Asp Ser Pro Lys Thr Tyr Val Gln Asp Ile Leu Arg
1081 1185 1190 1095
Thr Glu Leu Ala Ala Glu Val His Arg Val Leu Cys Leu Glu Arg
1096 1100 1105 1110
Gly His Met Phe Val Cys Gly Asp Val Thr Met Ala Thr Ser Val
1111 1115 1120 1125
Leu Gln Thr Val Gln Arg Ile Leu Ala Thr Glu Gly Asp Met Glu
1126 1130 1135 1140
Leu Asp Glu Ala Gly Asp Val Ile Gly Val Leu Arg Asp Gln Gln
1141 1145 1150 1155
Arg Tyr His Glu Asp Ile Phe Gly Leu Thr Leu Arg Thr Gln Glu
1156 1160 1165 1170
Val Thr Ser Arg Ile Arg Thr Gln Ser Phe Ser Leu Gln Glu Arg
1171 1175 1180 1185
His Leu Arg Gly Ala Val Pro Trp Ala Phe Asp Pro Pro Gly Pro
1186 1190 1195 1200
Asp Thr Pro Gly Pro
1201 1205
```

Polyclonal antibodies to NO synthase may be obtained using the whole molecule of human endothelial NO synthase of the following sequence:

```
SEQ. ID. NO. 2
Met Gly Asn Leu Lys Ser Val Ala Gln Glu Pro Gly Pro Pro Cys
  1 5 10 15
Gly Leu Gly Leu Gly Leu Gly Leu Gly Leu Cys Gly Lys Gln Gly
  16 20 25 30
Pro Ala Thr Pro Ala Pro Glu Pro Ser Arg Ala Pro Ala Ser Leu
  31 35 40 45
Leu Pro Pro Ala Pro Glu His Ser Pro Pro Ser Ser Pro Leu Thr
  46 50 55 60
Gln Pro Pro Glu Gly Pro Lys Phe Pro Arg Val Lys Asn Trp Glu
  61 65 70 75
Val GLys er Ile Thr Tyr Asp Thr Leu Ser Ala Gln Ala Gln Gln
  76 80 85 90
Asp Gly Pro Cys Thr Pro Arg Arg Cys Leu GLys er Leu Val Phe
  91 95 100 105
Pro Arg Lys Leu Gln Gly Arg Pro Ser Pro Gly Pro Pro Ala Pro
106 110 115 120
Glu Gln Leu Leu Ser Gln Ala Arg Asp Phe Ile Asn Gln Tyr Tyr
121 125 130 135
Ser Ser Ile Lys Arg Ser GLys er Gln Ala His Glu Gln Arg Leu
136 140 145 150
Gln Glu Val Glu Ala Glu Val Ala Ala Thr Gly Thr Tyr Gln Leu
151 155 160 165
Arg Glu Ser Glu Leu Val Phe Gly Ala Lys Gln Ala Trp Arg Asn
166 170 175 180
Ala Pro Arg Cys Val Gly Arg Ile Gln Trp Gly Lys Leu Gln Val
181 185 190 195
Phe Asp Ala Arg Asp Cys Arg Ser Ala Gln Glu Met Phe Thr Tyr
196 200 205 210
Ile Cys Asn His Ile Lys Tyr Ala Thr Asn Arg Gly Asn Leu Arg
211 215 220 225
Ser Ala Ile Thr Val Phe Pro Gln Arg Cys Pro Gly Arg Gly Asp
226 230 235 240
Phe Arg Ile Trp Asn Ser Gln Leu Val Arg Tyr Ala Gly Tyr Arg
241 245 250 255
Gln Gln Asp GLy Ser Val Arg Gly Asp Pro Ala Asn Val Glu Ile
256 260 265 270
Thr Glu Leu Cys Ile Gln His Gly Trp Thr Pro Gly Asn Gly Arg
271 275 280 285
Phe Asp Val Leu Pro Leu Leu Leu Gln Ala Pro Asp Glu Pro Pro
286 290 295 300
Glu Leu Phe Leu Leu Pro Pro Glu Leu Val Leu Glu Val Pro Leu
301 305 310 315
Glu His Pro Thr Leu Glu Trp Phe Ala Ala Leu Gly Leu Arg Trp
316 320 325 330
Tyr Ala Leu Pro Ala Val Ser Asn Met Leu Leu Glu Ile Gly Gly
331 335 340 345
Leu Glu Phe Pro Ala Ala Pro Phe Ser Gly Trp Tyr Met Ser Thr
346 350 355 360
Glu Ile Gly Thr Arg Asn Leu Cys Asp Pro His Arg Tyr Asn Ile
361 365 370 375
Leu Glu Asp Val Ala Val Cys Met Asp Leu Asp Thr Arg Thr Thr
376 380 385 390
Ser Ser Leu Trp Lys Asp Lys Ala Ala Val Glu Ile Asn Val Ala
391 395 400 405
Val Leu His Ser Tyr Gln Leu Ala Lys Val Thr Ile Val Asp His
406 410 415 420
His Ala Ala Thr Ala Ser Phe Met Lys His Leu Glu Asn Glu Gln
421 425 430 435
Lys Ala Arg Gly Gly Cys Pro Ala Asp Trp Ala Trp Ile Val Pro
436 440 445 450
Pro Ile Ser GLys er Leu Thr Pro Val Phe His Gln Glu Met Val
451 455 460 465
Asn Tyr Phe Leu Ser Pro Ala Phe Arg Tyr Gln Pro Asp Pro Trp
466 470 475 480
Lys Gly Ser Ala Ala Lys Gly Thr Gly Ile Thr Arg Lys Lys Thr
481 485 490 495
Phe Lys Glu Val Ala Asn Ala Val Lys Ile Ser Ala Ser Leu Met
496 500 505 510
Gly Thr Val Met Ala Lys Arg Val Lys Ala Thr Ile Leu Tyr Gly
511 515 510 525
Ser Glu Thr Gly Arg Ala Gln Ser Tyr Ala Gln Gln Leu Gly Arg
526 530 535 540
Leu Phe Arg Lys Ala Phe Asp Pro Arg Val Leu Cys Met Asp Glu
541 545 550 555
Tyr Asp Val Val Ser Leu Glu His Glu Thr Leu Val Leu Val Val
556 560 565 570
Thr Ser Thr Phe Gly Asn Gly Asp Pro Pro Glu Asn Gly Glu Ser
571 575 580 585
Phe Ala Ala Ala Leu Met Glu Met Ser Gly Pro Tyr Asn Ser Ser
586 590 595 600
Pro Arg Pro Glu Gln His Lys Ser Tyr Lys Ile Arg Phe Asn Ser
601 605 610 615
Ile Ser Cys Ser Asp Pro Leu Val Ser Ser Trp Arg Arg Lys Arg
616 620 625 630
Lys Glu Ser Ser Asn Thr Asp Ser Ala Gly Ala Leu Gly Thr Leu
631 635 640 645
Arg Phe Cys Val Phe Gly Leu GLys er Arg Ala Tyr Pro His Phe
646 650 655 660
Cys Ala Phe Ala Arg Ala Val Asp Thr Arg Leu Glu Glu Leu Gly
661 665 670 675
Gly Glu Arg Leu Leu Gln Leu Gly Gln Gly Asp Glu Leu Cys Gly
676 680 685 690
Gln Glu Glu Ala Phe Arg Gly Trp Ala Gln Ala Ala Phe Gln Ala
691 695 700 705
Ala Cys Glu Thr Phe Cys Val Gly Glu Asp Ala Lys Ala Ala Ala
706 710 715 720
Arg Asp Ile Phe Ser Pro Lys Arg Ser Trp Lys Arg Gln Arg Tyr
721 725 730 735
Arg Leu Ser Ala Gln Ala Glu Gly Leu Gln Leu Leu Pro Gly Leu
736 740 745 750
Ile His Val His Arg Arg Lys Met Phe Gln Ala Thr Ile Arg Ser
751 755 760 765
Val Glu Asn Leu Gln Ser Ser Lys Ser Thr Arg Ala Thr Ile Leu
766 770 775 780
Val Arg Leu Asp Thr Gly Gly Gln Glu Gly Leu Gln Tyr Gln Pro
781 785 790 795
Gly Asp His Ile Gly Val Cys Pro Pro Asn Arg Pro Gly Leu Val
796 800 805 810
Glu Ala Leu Leu Ser Arg Val Glu Asp Pro Pro Ala Pro Thr Glu
811 815 820 825
Pro Val Ala Val Glu Gln Leu Glu Lys Gly Ser Pro Gly Gly Pro
826 830 835 840
Pro Pro Gly Trp Val Arg Asp Pro Arg Leu Pro Pro Cys Thr Leu
841 845 850 855
Arg Gln Ala Leu Thr Phe Phe Leu Asp Ile Thr Ser Pro Pro Ser
856 860 865 870
Pro Gln Leu Leu Arg Leu Leu Ser Thr Leu Ala Glu Glu Pro Arg
871 875 880 885
Glu Gln Gln Glu Leu Glu Ala Leu Ser Gln Asp Pro Arg Arg Tyr
886 890 895 900
Glu Glu Trp Lys Trp Phe Arg Cys Pro Thr Leu Leu Glu Val Leu
901 905 910 915
Glu Gln Phe Pro Ser Val Ala Leu Pro Ala Pro Leu Leu Leu Thr
916 920 925 930
Gln Leu Pro Leu Leu Gln Pro Arg Tyr Tyr Ser Val Ser Ser Ala
931 935 940 945
Pro Ser Thr His Pro Gly Glu Ile His Leu Thr Val Ala Val Leu
946 950 955 960
Ala Tyr Arg Thr Gln Asp Gly Leu Gly Pro Leu His Tyr Gly Val
961 965 970 975
Cys Ser Thr Trp Leu Ser Gln Leu Lys Pro Gly Asp Pro Val Pro
976 980 985 990
Cys Phe Ile Arg Gly Ala Pro Ser Phe Arg Leu Pro Pro Asp Pro
991 995 1000 1005
Ser Leu Pro Cys Ile Leu Val Gly Pro Gly Thr Gly Ile Ala Pro
1006 1010 1015 1020
Phe Arg Gly Phe Trp Gln Glu Arg Leu His Asp Ile Glu Ser Lys
1021 1025 1030 1035
Gly Leu Gln Pro Thr Pro Met Thr Leu Val Phe Gly Cys Arg Cys
1036 1140 1145 1050
Ser Gln Leu Asp His Leu Tyr Arg Asp Glu Val Gln Asn Ala Gln
1051 1155 1160 1065
Gln Arg Gly Val Phe Gly Arg Val Leu Thr Ala Phe Ser Arg Glu
1066 1170 1175 1080
Pro Asp Asn Pro Lys Thr Tyr Val Gln Asp Ile Leu Arg Thr Glu
1081 1185 1190 1095
Leu Ala Ala Glu Val His Arg Val Leu Cys Leu Glu Arg Gly His
1096 1100 1105 1110
Met Phe Val Cys Gly Asp Val Thr Met Ala Thr Asn Val Leu Gln
1111 1115 1120 1125
Thr Val Gln Arg Ile Leu Ala Thr Glu Gly Asp Met Glu Leu Asp
1126 1130 1135 1140
Glu Ala Gly Asp Val Ile Gly Val Leu Arg Asp Gln Gln Arg Tyr
1141 1145 1150 1155
His Glu Asp Ile Phe Gly Leu Thr Leu Arg Thr Gln Glu Val Thr
1156 1160 1165 1170
Ser Arg Ile Arg Thr Gln Ser Phe Ser Leu Gln Glu Arg Gln Leu
1171 1175 1180 1185
Arg Gly Ala Val Pro Trp Ala Phe Asp Pro Pro Gly Ser Asp Thr
1186 1190 1195 1200
Asn Ser Pro
1201 1203
```

To obtain polyclonal antibodies to NO synthase, it is also possible to use a fragment of endothelial NO synthase, selected, for example, from the following sequences:

```
SEQ. ID. NO. 3
Pro Trp Ala Phe
1192 1195
SEQ. ID. NO. 4
Gly Ala Val Pro
1189 1192
SEQ. ID. NO. 5
                                                                                                                Arg
                                                                                                              1185
His Leu Arg Gly Ala Val Pro Trp Ala Phe Asp Pro Pro Gly Pro
1186 1190 1195 1200
Asp Thr Pro Gly Pro
1201 1205
SEQ. ID. NO. 6
                                                                Ala Phe Asp Pro Pro Gly Pro
                                                                11941195
1200
Asp Thr Pro Gly Pro
1201 1205
SEQ. NO. 7
His Leu Arg Gly Ala Val Pro Trp Ala Phe Asp
1186 1190 11951196
SEQ. ID. NO. 8
His Leu Arg Gly Ala Val Pro Trp Ala Phe Asp Pro Pro Gly Pro
1186 1190 1195 1200
Asp Thr Pro Gly Pro
1201 1205
```

The exemplary procedure for preparation of starting polyclonal antibodies to NO synthase may be described as follows: 7-9 days before blood sampling 1-3 intravenous injections are made to the rabbits to increase the level of polyclonal antibodies in the rabbit blood stream. Upon immunization, blood samples are taken to test the antibody level. Typically, the maximum level of the immune reaction of the soluble antigen is reached in 40-60 days after the first injection. After the termination of the first immunization cycle, rabbits have a 30-day rehabilitation period, after which re-immunization is performed with another 1-3 intravenous injections.

To obtain antiserum containing the desired antibodies, the immunized rabbits' blood is collected from rabbits and placed in a 50 ml centrifuge tube Product clots formed on the tube sides are removed with a wooden spatula, and a rod is placed into the clot in the tube center. The blood is then placed in a refrigerator for one night at the temperature of about 4° C. On the following day, the clot on the spatula is removed, and the remaining liquid is centrifuged for 10 min at 13,000 rotations per minute. Supernatant fluid is the target antiserum. The obtained antiserum is typically yellow. 20% of NaN₃ (weight concentration) is added in the antiserum to a final concentration of 0.02% and stored before use in frozen state at the temperature of −20° C. (or without addition NaN₃—at temperature −70° C.). To separate the target antibodies to endothelial NO synthase from the antiserum, the following solid phase absorption sequence is suitable:

(a) 10 ml of antiserum of rabbit is diluted twofold with 0.15 M NaCl, after which 6.26 g Na₂SO₄, is added, mixed and incubated for about 12-16 hours at 4° C.;

(b) the sediment is removed by centrifugation, dissolved in 10 ml of phosphate buffer and dialyzed against the same buffer within one night at room temperature;

(c) after the sediment is removed by centrifugation, the solution is put on the column with DEAE-cellulose, counterbalanced by phosphate buffer;

(d) the antibody fraction is determined by measuring the optical density of eluate at 280 nanometers.

The isolated crude antibodies are purified using affine chromatography method by attaching the obtained antibodies to endothelial NO synthase located on the insoluble matrix of the chromatography media, with subsequent elution by concentrated aqueous salt solutions.

The resulting buffer solution is used as the initial solution for the homeopathic dilution process used to prepare the activated potentiated form of the antibodies. The preferred concentration of the initial matrix solution of the antigen-purified polyclonal rabbit antibodies to endothelial NO synthase is 0.5 to 5.0 mg/ml, preferably, 2.0 to 3.0 mg/ml.

The brain-specific S100 protein, expressed by neurons and glial cells (astrocytes and oligodendrocytes), directly or through interactions with other proteins executes in the CNS a number of functions directed at maintaining normal brain functioning, including affecting learning and memory processes, growth and viability of neurons, regulation of metabolic processes in neuronal tissues and others. To obtain polyclonal antibodies to brain-specific protein S-100, brain-specific protein S-100 is used, which physical and chemical properties are described in the article of M. V. Starostin, S. M. Sviridov, Neurospecific Protein S-100, Progress of Modern Biology, 1977, Vol. 5, P. 170-178; found in the book M. B. Shtark, Brain-Specific Protein Antigenes and Functions of Neuron, “Medicine”, 1985; P. 12-14. Brain-specific protein S-100 is allocated from brain tissue of the bull by the following technique:
the bull brain tissue frozen in liquid nitrogen is converted into powder using a specialized mill;
proteins are extracted in the ratio of 1:3 (weight/volume) using an extracting buffer with homogenization;
the homogenate is heated for 10 min at 60° C. and then cooled to 4° C. in an ice bath;
thermolabile proteins are removed by centrifugation;
ammonium sulfate fractionation is carried out in stages, with subsequent removal of precipitated proteins;
the fraction containing S-100 protein is precipitated using 100% saturated ammonium sulfate accomplished by pH drop to 4.0; the desired fraction is collected by centrifugation;
the precipitate is dissolved in a minimum buffer volume containing EDTA and mercaptoethanol, the precipitate is dialyzed with deionized water and lyophilized;
fractionation of acidic proteins is followed by chromatography in ion-exchanging media, DEAE-cellulose DE-52 and then DEAE-sephadex A-50;
the collected and dialyzed fractions, which contain S-100 protein, are divided according to molecular weight by gel filtration on sephadex G-100;

purified S-100 protein is dialyzed and lyophilized.

The molecular weight of the purified brain-specific protein S-100 is 21000 D.

Owing to the high concentration of asparaginic and glutaminic acids brain-specific protein S-100 is highly acidic and occupies extreme anode position during electroendosmosis in a discontinuous buffer system of polyacrylamide gel which facilitates its identification.

The polyclonal antibodies to S-100 protein may also be obtained by a similar methodology to the methodology described for endothelial NO synthase antibodies using an adjuvant. The entire molecule of S-100 protein may be used as immunogen (antigen) for rabbits' immunization:

```
Bovine S100B
(SEQ. ID. NO. 9)
Met Ser Glu Leu Glu Lys Ala Val Val Ala Leu Ile Asp Val Phe
  1 5 10 15
His Gln Tyr Ser Gly Arg Glu Gly Asp Lys His Lys Leu Lys Lys
  16 20 25 30
Ser Glu Leu Lys Glu Leu Ile Asn Asn Glu Leu Ser His Phe Leu
  31 35 40 45
Glu Glu Ile Lys Glu Gln Glu Val Val Asp Lys Val Met Glu Thr
  46 50 55 60
Leu Asp Ser Asp Gly Asp Gly Glu Cys Asp Phe Gln Glu Phe Met
  61 65 70 75
Ala Phe Val Ala Met Ile Thr Thr Ala Cys His Glu Phe Phe Glu
  76 80 85 90
His Glu
  91 92
Human S100B
(SEQ. ID. 10)
Met Ser Glu Leu Glu Lys Ala Met Val Ala Leu Ile Asp Val Phe
  1 5 10 15
His Gln Tyr Ser Gly Arg Glu Gly Asp Lys His Lys Leu Lys Lys
  16 20 25 30
Ser Glu Leu Lys Glu Leu Ile Asn Asn Glu Leu Ser His Phe Leu
  31 35 40 45
Glu Glu Ile Lys Glu Gln Glu Val Val Asp Lys Val Met Glu Thr
  46 50 55 60
Leu Asp Asn Asp Gly Asp Gly Glu Cys Asp Phe Gln Glu Phe Met
  61 65 70 75
Ala Phe Val Ala Met Val Thr Thr Ala Cys His Glu Phe Phe Glu
  76 80 85 90
His Glu
  91 92
Human S100A1
(SEQ. ID. No. 11)
Met Gly Ser Glu Leu Glu Thr Ala Met Glu Thr Leu Ile Asn Val
  1 5 10 15
Phe His Ala His Ser Gly Lys Glu Gly Asp Lys Tyr Lys Leu Ser
  16 20 25 30
Lys Lys Glu Leu Lys Glu Leu Leu Gln Thr Glu Leu Ser Gly Phe
  31 35 40 45
Leu Asp Ala Gln Lys Asp Val Asp Ala Val Asp Lys Val Met Lys
  46 50 55 60
Glu Leu Asp Glu Asn Gly Asp Gly Glu Val Asp Phe Gln Glu Tyr
  61 65 70 75
Val Val Leu Val Ala Ala Leu Thr Val Ala Cys Asn Asn Phe Phe
  76 80 85 90
Trp Glu Asn Ser
  91 94
Bovine S100A1
(SEQ. ID. NO. 12)
Met Gly Ser Glu Leu Glu Thr Ala Met Glu Thr Leu Ile Asn Val
  1 5 10 15
Phe His Ala His Ser Gly Lys Glu Gly Asp Lys Tyr Lys Leu Ser
  16 20 25 30
Lys Lys Glu Leu Lys Glu Leu Leu Gln Thr Glu Leu Ser Gly Phe
  31 35 40 45
Leu Asp Ala Gln Lys Asp Ala Asp Ala Val Asp Lys Val Met Lys
  46 50 55 60
Glu Leu Asp Glu Asn Gly Asp Gly Glu Val Asp Phe Gln Glu Tyr
  61 65 70 75
Val Val Leu Val Ala Ala Leu Thr Val Ala Cys Asn Asn Phe Phe
  76 80 85 90
Trp Glu Asn Ser
  91 94
```

To obtain antiserum, brain-specific S-100 protein or the mixture of S-100 protein s (antigens) in complex with methylated bull seralbumin as the carrying agent with full Freund's adjuvant is prepared and added to allocated brain-specific protein S-100 which is injected subdermally to a laboratory animal—a rabbit into area of back in quantity of 1-2 ml. On 8th, 15th day repeated immunization is made. Blood sampling is made (for example, from a vein in the ear) on the 26th and the 28th day.

The obtained antiserum titre is 1:500-1:1000, forms single precipitin band with an extract of nervous tissue but does not react with extracts of heterological bodies and forms single precipitin peak both with pure protein S-100 and with the extract of nervous tissue indicating that the antiserum obtained is monospecific.

The activated potentiated form of each component of the combination may be prepared from an initial solution by homeopathic potentization, preferably using the method of proportional concentration decrease by serial dilution of 1 part of each preceding solution (beginning with the initial solution) in 9 parts (for decimal dilution), or in 99 parts (for centesimal dilution), or in 999 parts (for millesimal dilution—attenuation M) of a neutral solvent, starting with a concentration of the initial solution of antibody in the solvent, preferably, water or a water-ethyl alcohol mixture, in the range from about 0.5 to about 5.0 mg/ml, coupled with external impact. Preferably, the external impact involves multiple vertical shaking (dynamization) of each dilution. Preferably, separate containers are used for each subsequent dilution up to the required potency level, or the dilution factor. This method is well-accepted in the homeopathic art. See, e.g. V. Schwabe “Homeopathic medicines”, M., 1967, p. 14-29, incorporated herein by reference for the purpose stated.

For example, to prepare a 12-centesimal dilution (denoted C12), one part of the initial matrix solution of antibodies to brain-specific protein S-100 (or to endothelial NO-synthase) with the concentration of 2.5 mg/ml is diluted in 99 parts of neutral aqueous or aqueous-alcohol solvent (preferably, 15%-ethyl alcohol) and then vertically shaken many times (10 and more) to create the 1st centesimal dilution (denoted as C1). The 2nd centesimal dilution (C2) is prepared from the 1st centesimal dilution C1. This procedure is repeated 11 times to prepare the 12th centesimal dilution C12. Thus, the 12th centesimal dilution C12 represents a solution obtained by 12 serial dilutions of one part of the initial matrix solution of antibodies to brain-specific protein S-100 with the concentration of 2.5 mg/ml in 99 parts of a neutral solvent in different containers, which is equivalent to the centesimal homeopathic dilution C12. Similar procedures with the relevant dilution factor are performed to obtain dilutions C30, C50 and C 200. The intermediate dilutions may be tested in a desired biological model to check activity. The preferred activated potentiated forms for both antibodies comprising the combination of the invention are a mixture of C12, C30, and C200 dilutions or C12, C30 and C50 dilutions. When using the mixture of various homeopathic dilutions (primarily centesimal) of the active substance as biologically active liquid component, each component of the composition (e.g., C12, C30, C50, C200) is prepared separately according to the above-described procedure until the next-to-last dilution is obtained (e.g., until C11, C29, C49 and C199 respectively), and then one part of each component is added in one container according to the mixture composition and mixed with the required quantity of the solvent (e.g. with 97 parts for centesimal dilution).

Thus, activated-potentiated form of antibodies to brain-specific protein S-100 in ultra low dose is obtained by extra attenuation of matrix solution, accordingly in 100¹², 100³⁰ and 100²⁰⁰ times, equal to centesimal C12, C30 and C200 solutions or 100¹², 100³⁰ and 100⁵⁰ times, equal to centesimal C12, C30 and C50 solutions prepared on homoeopathic technology.

Use of active substance in the form of mixture of other various solutions on homoeopathic technology, for example, decimal and/or centesimal, (C12, C30, C100; C12, C30, C50; D20, C30, C100 or D10, C30, M100 etc.) is possible. The efficiency is defined experimentally.

External processing in the course of potentiation and concentration reduction can also be carried out by means of ultrasound, of electromagnetic or any other physical influence accepted in the homeopathic art.

Preferably, the combination pharmaceutical composition of the invention may be in the form of a liquid or in the solid unit dosage form. The preferred liquid form of the pharmaceutical composition is a mixture, preferably, at a 1:1 ratio of the activated potentiated form of antibodies to endothelial NO synthase and the activated potentiated form of antibodies to protein S-100. The preferred liquid carrier is water or water-ethyl alcohol mixture.

The solid unit dosage form of the pharmaceutical composition of the invention may be prepared by using impregnating a solid, pharmaceutically acceptable carrier with the mixture of the activated potentiated form aqueous or aqueous-alcohol solutions of active components that are mixed, primarily in 1:1 ratio and used in liquid dosage form. Alternatively, the carrier may be impregnated consecutively with each requisite dilution. Both orders of impregnation are acceptable.

Preferably, the pharmaceutical composition in the solid unit dosage form is prepared from granules of the pharmaceutically acceptable carrier which was previously saturated with the aqueous or aqueous-alcoholic dilutions of the activated potentiated form of antibodies. The solid dosage form may be in any form known in the pharmaceutical art, including a tablet, a capsule, a lozenge, and others. As an inactive pharmaceutical ingredients one can use glucose, sucrose, maltose, amylum, isomaltose, isomalt and other mono- olygo- and polysaccharides used in manufacturing of pharmaceuticals as well as technological mixtures of the above mentioned inactive pharmaceutical ingredients with other pharmaceutically acceptable excipients, for example isomalt, crospovidone, sodium cyclamate, sodium saccharine, anhydrous citric acid etc), including lubricants, disintegrants, binders and coloring agents. The preferred carriers are lactose and isomalt. The pharmaceutical dosage form may further include standard pharmaceutical excipients, for example, microcrystalline cellulose, magnesium stearate and citric acid.

The example of preparation of the solid unit dosage form is set forth below. To prepare the solid oral form, 100-300 μm granules of lactose are impregnated with aqueous or aqueous-alcoholic solutions of the activated potentiated form of antibodies to histamine, activated-potentiated form of antibodies to endothelial NO synthase and the activated potentiated form of antibodies to protein S-100 in the ratio of 1 kg of antibody solution to 5 or 10 kg of lactose (1:5 to 1:10). To effect impregnation, the lactose granules are exposed to saturation irrigation in the fluidized boiling bed in a boiling bed plant (e.g. “Hüttlin Pilotlab” by Hüttlin GmbH) with subsequent drying via heated air flow at a temperature below 40° C. The estimated quantity of the dried granules (10 to 34 weight parts) saturated with the activated potentiated form of antibodies is placed in the mixer, and mixed with 25 to 45 weight parts of “non-saturated” pure lactose (used for the purposes of cost reduction and simplification and acceleration of the technological process without decreasing the treatment efficiency), together with 0.1 to 1 weight parts of magnesium stearate, and 3 to 10 weight parts of microcrystalline cellulose. The obtained tablet mass is uniformly mixed, and tableted by direct dry pressing (e.g., in a Korsch-XL 400 tablet press) to form 150 to 500 mg round pills, preferably, 300 mg. After tableting, 300 mg pills are obtained that are saturated with aqueous-alcohol solution (3.0-6.0 mg/pill) of the combination of the activated-potentiated form of antibodies. Each component of the combination used to impregnate the carrier is in the form of a mixture of centesimal homeopathic dilutions, preferably, C12, C30 and C200.

Preferably, 1-2 tablets of the claimed pharmaceutical composition are administered 2-4 times a day.

The combination of activated-potentiated form of antibodies to brain specific protein S-100 and activated-potentiated form of antibodies to endothelial NO-synthase in pharmaceutical composition are prepared according to homeopathic technology of exponentiation through repeated subsequent dilution in combination with external mechanical effect—vertical shaking of every dilution (see, for example, V. Shwabe “Homeopathic drugs”, M., 1967, p. 14-29) that possess activity caused by the technology of exponentiation within pharmacological models and/or clinical methods of treatment of vertigo of various genesis, kinetosis and vegetative-vascular dystonia) provides obtaining of sudden synergetic therapeutic effect confirmed on adequate (valid) experimental models and clinical investigations that consists in increase of efficiency of treatment of both vertigo of various genesis, kinetosis and vegetative-vascular dystonia. The mentioned technical result is provided by enhancement of neuroprotective activity of antibodies to protein S-100 caused by influence on efficiency of interaction of ligands with sigma-1 receptor, vegetative stabilizing effect, normalization of vegetative status as through manifestation of earlier non-exposed features of activated-potentiated form of antibodies to brain specific protein S-100 and synergetic influence of both components on neutral plasticity and as a result of it through increase of resistance of brain to toxic effects that improves integrative activity and restores interhemispheric relations of brain, facilitates elimination of cognitive disorders, stimulates reparative processes and accelerates restoration of function of stabilizes somatovegetative manifestations, increases cerebral blood flow and, respectively, provides enlargement of therapeutic range of medication and increase of efficiency of treatment of vertigo, kinetosis and vegetative-vascular dystonia of various genesis including vegetative-vascular dystonia accompanied by both increase and decrease of blood pressure. Moreover the declared drug and its components do not possess sedative and miorelaxation effect, do not evoke addiction and adaptation. The declared drug can also be used as the component of complex therapy.

Moreover the declared drug broadens assortment of medications designed for the treatment of vertigo of various genesis, kinetosis and vegetative-vascular dystonia.

In addition, the combination pharmaceutical composition of the present invention may be used for the treatment of attention deficit hyperactivity disorder, psychoorganic syndrome, encephalopathies of different origin, organic diseases of nervous system, including stroke, Alcheimer's disease, Parkinson's disease. For the treatment of said disorders the combination pharmaceutical composition may contain active components in volume ratio 1:1, thus, each component is used as the mixture of three matrix solutions (mother tincture) of antibodies diluted 100¹², 100³⁰ and 100²⁰⁰ times, respectively, which is equivalent to centesimal homeopathic dilutions (C12, C30, and C200) or mixture of three matrix solutions of antibodies diluted 100¹², 100³⁰ and 100⁵⁰ times, respectively, which is equivalent to centesimal homeopathic dilutions (C12, C30 and C50). The claimed pharmaceutical composition is recommended to be taken, preferably in 1-2 tablets 2-6 times (preferably 2-4 times) a day.

The claimed pharmaceutical composition as well as its components does not possess sedative and myorelaxant effect, does not cause addiction and habituation.

### EXAMPLES

### Example 1

Study of the effect of a complex preparation containing ultralow doses of activated-potentiated forms of polyclonal affinity purified rabbit antibodies to brain-specific protein S-100 (anti-S100) and endothelial NO-synthase (anti-eNOS), obtained by super-dilution of initial matrix solution (concentration: 2.5 mg/ml) (100¹², 100³⁰, 100²⁰⁰ times), equivalent to a mixture of centesimal homeopathic dilutions C12, C30, C200 (ratio: 1:1) (“ULD of anti-S100+anti-eNOS”), as well as its components: activated-potentiated form of polyclonal affinity purified rabbit antibodies to ultralow doses of brain-specific protein S-100, purified on antigen, obtained by super-dilution of initial matrix solution (100¹², 100³⁰, 100²⁰⁰ times, equivalent to a mixture of centesimal homeopathic dilution C12, C30, C200 (“ULD of anti-S100”), and activated-potentiated form of polyclonal rabbit antibodies to ultralow dose of endothelial NO-synthase, obtained by super-dilution of initial matrix solution (100¹², 100³⁰, 100²⁰⁰ times), equivalent to a mixture of centesimal homeopathic dilution C12, C30, C200 (“ULD of anti-eNOS”) on in vitro on binding of standard ligand [³H]pentazocine to human recombinant σ1 receptor was evaluated using radioligand method. Potentiated distilled water (mixture of homeopathic dilutions C12+C30+C200) was used as test preparations control.

The sigma-1 (σ1) receptor is an intracellular receptor which is localized in the cells of central nervous system, the cells of the most of peripheral tissues and immune component cells. These receptors exhibit a unique ability to be translocated which is thought to be caused by many psychotropic medications. The dynamics of sigma-1 receptors is directly linked to various influences which are performed by preparations acting to the sigma-1 receptors. These effects include, the regulation of activity channels, ecocytosis, signal transferring, remodeling of the plasma membrane (formation of rafts) and lipid transportation/metabolism, all of which can contribute to the plasticity of neurons in a brain. There is evidence that the sigma-1 receptors have a modulating effect on all the major neuromediator systems: noradrenergic, serotonergic, dopaminergic, cholinergic systems and NMDA-adjustable glutamate effects. Sigma-1 receptors play an important role in the pathophysiology of neurodegenerative diseases (e.g., Alzheimer's disease, Parkinson's disease), psychiatric and affective disorders and stroke; and they also take part in the processes of learning and memory. In this regard, the ability of drugs to influence the efficiency of interaction of ligands with sigma-1 receptor is indicative of the presence of neuroprotective, anti-ischemic, anxiolytic, antidepressant and anti astenic components in the spectrum of its pharmacological activity and permits the consideration of these drugs as effective preparations particularly for the treatment of cerebrovascular diseases.

During the test (to measure total binding) 20 μl of the complex preparation of ULD of anti-S100+anti-eNOS or 10 μl of ULD of anti-S100 or 10 μl of ULD of anti-NOS were added to the incubation medium. Thus, the quantity of ULD of anti-S100+anti-eNOS transferred into the test basin when testing the complex preparation was identical to that of ULD of anti-S100 and ULD of anti-NOS tested as monopreparations, which allows for a comparison of the efficiency of the preparation to its separate components. 20 μl and 10 μl of potentiated water were transferred into the incubation medium.

Further, 160 μl (about 200 μg of protein) of Jurkat cell line membranes homogenate (human leukemic T-lymphocyte line), and finally, 20 μl of tritium-labeled radioligand [³H]pentazocine (15 nm) were transferred.

In order to measure non-specific binding, 20 μl of non-labeled ligand-haloperidol (10 μM) were transferred in the incubation medium instead of the preparations or potentiated water.

Radioactivity was measured using a scintillometer (Topcount, Packard) and scintillation blend (Microscint 0, Packard) following the incubation within 120 minutes at 22° C. in 50 mM Tris-HCl buffer (pH=7.4) and filtration using fiberglass filters (GF/B, Packard). Specific binding (during the test or control) was calculated as a difference between total (during the test or control) and non-specific binding.

Results are represented as percentage of specific binding inhibition in control (distilled water was used as control) (Table 1).

TABLE 1

% of
radioligand

| | |
|---|---|
| % of radioligand specific | binding |

| | Quantity per | binding in control | | | inhibition in |
|---|---|---|---|---|---|
| Test group | test basin | 1^{st} test | 2^{nd} test | Average | control |
| ULD of | 20 μl | 48.4 | 35.5 | 42.0 | 58.0 |
| anti-S100+ | | | | | |
| anti-eNOS | | | | | |
| ULD of | 10 μl | 67.3 | 63.1 | 65.2 | 34.8 |
| anti-S100 | | | | | |
| ULD of | 10 μl | 147.5 | 161.1 | 154.3 | −54.3 |
| anti-eNOS | | | | | |
| Potentiated | 20 μl | 98.1 | 75.8 | 86.9 | 13.1 |
| water | | | | | |
| Potentiated | 10 μl | 140.1 | 106.2 | 123.2 | −23.2 |
| water | | | | | |

Effect of the preparations and potentiated water on binding of standard ligand [³H]pentazocine to human recombinant σ 1 receptor
Note:
% of specific binding in control = (specific binding during the test/specific binding in control)* 100%;
% of specific binding inhibition in control = 100% - (specific binding during the test/specific binding in control) * 100%).

The results reflecting inhibition above 50% represents significant effects of the tested compounds; inhibition from 25% to 50% confirms mild to moderate effects; inhibition less than 25% is considered to be insignificant effect of the tested compound and is within background level.

Therefore, this test model showed that the complex preparation of ULD of anti-S100+anti-eNOS is more efficient than its separate components (ULD of anti-S100 and ULD of anti-eNOS) in inhibiting the binding of standard radioligand [³H]pentazocine to human recombinant σ1 receptor; ULD of anti-S100, transferred into the test basin, namely 10 μl, inhibit the binding of standard radioligand [³H]pentazocine to human recombinant σ1 receptor, but the effect intensity is inferior to that of the complex preparation of ULD of anti-S100+anti-eNOS; ULD of anti-eNOS, transferred into the test well, namely 10 μl, had no effect on the binding of standard radioligand [3H]pentazocine to human recombinant σ1 receptor; potentiated water, transferred into the test basin, namely 10 μl or 20 μl, had no effect on the binding of standard radioligand [3H]pentazocine to human recombinant σ1 receptor.

### Example 2

The following preparation were used: 300 mg tablets impregnated with aqueous alcoholic solution (3 mg/tab) of activated-potentiated form of polyclonal rabbit antibodies to brain specific S-100 protein (anti-S-100), purified on an antigen, in ultra low dose (ULD anti-S100) obtained by super dilution of initial solution (with concentration of 2.5 mg/ml) in 100¹², 100³⁰, 100²⁰⁰ times, of equivalent mixture of centesimal homeopathic dilutions C12, C30, C200; 300 mg tablets impregnated with aqueous-alcohol solutions of (6 mg/tab) of activated-potentiated forms of polyclonal affinity purified rabbit antibodies to brain-specific protein S-100 (anti S-100) and to eNOS (anti-eNOS) in ultra low dose (ULD anti-S-100+ULD anti-eNOS), obtained by super dilution of initial solution (with concentration of 2.5 mg/ml) in 100¹², 100³⁰, 100²⁰⁰ times, of equivalent mixture of centesimal homeopathic dilutions C12, C30, C200; 300 mg tablets impregnated with aqueous-alcohol solution (3 mg/tab) of activated-potentiated form of polyclonal rabbit anti-eNOS purified on an antigen in ultra low dose (ULD anti-eNOS), obtained by super dilution of initial solution (with concentration of 2.5 mg/ml) in 100¹², 100³⁰, 100²⁰⁰ times, of equivalent mixture of centesimal homeopathic dilutions C12, C30, C200; and as placebo 300 mg tablets containing excipients: lactose (lactose monohydrate)—267 mg, microcrystal cellulose—30 mg, magnesium stearate—3 mg.

The effectiveness of the studied drugs in the treatment of dizziness (vertigo) and other symptoms of motion sickness was evaluated on kinetosis model or motion diseases/motion sicknesses which occurs by various vestibular vegetative disorders. Dizziness is the typical sign of lesion of the vestibular analyzer of various genesis including dysfunction of the vestibular nerve and cochlear system, circulatory embarrassment in vertebral basilar system, pathology of the central nervous system (CNS), etc. Dizziness as a manifestation of kinetosis accompanied with other vestibular-vegetative disorders which include three types of reactions: the vestibular-motor (nystagmus and the reaction of deviation), vestibular-sensory (in addition to dizziness, nystagmus is (or reaction of post rotation), defensive movements) and vegetative (nausea, vomiting, sweating, palpitation, heat feeling, pulse and blood pressure fluctuations).

Double blind placebo controlled comparative study were conducted in parallel groups consisting of 15 somatically healthy subjects—males and females aged from 15 to 60 years (mean age 33.3±0.75 years) with low (n=5; 33%) or mean (n=10; 67%) degree of motion sickness resistance in order to test anti motion sickness properties of various compositions. Group I was given ULD anti-S100+anti-eNOS, Group 2 was given ULD anti-S100 and Group 3 was given anti-eNOS.

To simulate the condition of motion sickness and evaluate the effectiveness of studied drugs the most appropriate and recognized kinetosis models—test with a continuous cumulative effect of accelerations by Coriolis (CCEAC) was used. Initial tolerance of CCEAC test in all study subjects was not more than 5 minutes. Vestibular-vegetative disorders provoked by kinetic effect (CCEAC) were registered with usage of complex of diagnostic methods including subject's examination, quantitative evaluation of disorders of vestibular-vegetative sensitivity (Halle scale), analysis of heart rate variability (HRV), and self-esteem of functional condition (WBAM—well-being, activity, and mood). As the criteria of efficiency of conducted therapy the dynamics of tolerance and extent of recovery period at kinetic influence were assessed as well as alteration of indexes' evidence of sensory-motor reactions (nystagmus), HRV indexes (with usage of Biocom Wellness Scan system, developed by AWS, LLC in accordance with International Standard of European Cardiologists Association and North American Electrophysiology Association) and WBAM data. The safety criteria were character, evidence and terms of emergence of probable adverse events (AE) in the treatment period connected with medication intake; influence of studied drugs for indexes which characterize the function of central nervous system (CNS) (reaction on moving object (RMO)), the time of simple motor reaction (TSMR); the dynamics of physical and functional factors (heart rate (HR), systolic and diastolic blood pressure (SBP, DBP), Stange's test; exercise tolerance (index of Harvard step-test). Safety was assessed after single dose administration and after 7-day course administration of the combination ULD anti-S-100 and ULD anti-eNOS.

All the subjects during 1 month before being involved into the study had not taken any drugs. After screening the subjects were randomized into 4 groups (Group 1—ULD anti-S100+anti-eNOS, Group 2—ULD anti-S100, Group 3—ULD anti-eNOS, and Group 4—placebo).

On the first day of the study (Visit 1) the initial functional and psychophysiological state of the subjects was registered, the subjects were then given 5 tablets of the respective ULD antibodies, followed by administration of the CCEAC test. The duration of the test was registered; vegetative-vestibular disorders and AEs related to motion sickness were detected with the help of a complex diagnostic examination. In the next 2-6 days the subject were given 1 tablet three times a day of the prescribed drug. At the 7^{th} day (Visit 2) the subjects were given the same dosage as on the first day (Visit 1). The complex of diagnostic studies was conducted before and after the CCEAC test. The study was organized in such way as study crew would work only with one subject. The study was parallel and conducted in the first half of a day with participation of, as a rule, 4 persons in a day, one person for drug or placebo. The next three weeks were washout period, at the end of which the new drug or placebo was prescribed to subjects of each group; the cycle of study was being repeated (Visit 1, the course intake of a drug; Visit 2). Thus, during the study each subject took part in four cycles of study. That is, each subject participated in each group with a three-week washout period between each cycle. This allowed the researcher to level the influence of individual peculiarities of a test person on the treatment effect. The analysis of drug efficiency was conducted on the data of all the test subjects who has completed the full course of studied drug intake according to study protocol (n=15).

The evidence factors of symptoms of motion sickness (vertigo, nausea, inactivity, skin pallor, sweatiness, etc.) after kinetic influence (CCEAC) against the background of single-day intake of studied drugs evidenced that all the study subjects have gained roughly the same state of motion sickness as far as the evidence of assessed symptoms of vegetative dysfunction on Halle's scale by physician-researcher was not differed significantly in all groups (table 2, Visit 1). However, while the kinetic affect which cause similar symptoms of motion sickness was different in four groups and was dependent on the drug which was taken by the subjects of the study (Table 3, Visit 1). One-day intake of ULD anti-S100+anti-eNOS preparation led to most clearly anti motion sickness effect which manifested itself not only in significantly more time of tolerance of CCEAC test (104.10±13.14 sec. vs. 68.50±6.57 sec.—in the group of ULD anti-S100; 75.00±6.79 sec.—in the group of ULD anti-eNOS and 61.30±3.15 sec.—in the placebo group) but also in the least time of nystagmus (9.90±1.20 sec. vs. 13.50±1.51; 16.10±1.68 and 13.30±1.12 sec., respectively) and in maximal rapid recovery (96.90±13.54 sec. vs. 194.20±18.45; 202.50±21.72 and 241.70±38.41 sec., respectively).

Roughly similar indexes were registered at Visit 2 after receiving a course of drugs. To achieve the similar symptoms of motion sickness (Table 2, Visit 2) the longest time of kinetic impact was applied to the subjects who has been receiving the composition of ULD anti-S100+anti-eNOS (Table 3, Visit 2) for 7 days. The most pronounced anti motion sickness effect of the composition of ULD anti-S100+anti-eNOS was expressed in significantly less time of nystagmus (9.50±1.38 sec, p<0.01) and duration of the recovery period (117.90±15.65 sec; p<0.01). The monocomponent preparation ULD anti-S100 had anti motion sickness action as better indexes of tolerance of CCEAC test, recovery time of nystagmus and recovery than in the placebo group evidenced (Table 3, Visits 1 and 2), but the efficacy of ULD anti-S100 was inferior to composition of ULD anti-S100+anti-eNOS. The monocomponent preparation ULD anti-eNOS did not show anti motion sickness effect since the results of CCEAC tests and subsequent recovery period had no significant difference from the placebo group (Table 3, Visits 1 and 2). Comparative analysis of indexes of CCEAC test in the groups of ULD anti-S100+anti-eNOS and ULD anti-S100 in one-day intake of the drugs has shown that the addition of ULD anti-eNOS increased the tolerance of the kinetic effect on the 52%, reduced the nystagmus time on 27% and contributed to the reduction the recovery period after the end of the kinetic effect on 50% including the duration of dizziness—on 49%. However, the greatest contribution of the component of ULD anti-eNOS introduced the effectiveness of combined preparation (compositions of ULD anti-S100+anti-eNOS) in course intake of a drug which was expressed in excess of 30% of the result achieved in the group of ULD anti-S100 by factors of tolerance of kinetic effect and nystagmus duration (in each of the parameters). In addition, the growth of the effect on Visit 2 by indexes of tolerance of CCEAC test and duration of the nystagmus in relation to data of Visit 1 when taking the composition ULD anti-S100+anti-eNOS in comparison to monocomponent preparation ULD anti-S100 was expressed in a greater degree as confirmed by alteration of these indexes on 30% and 4% (versus 21% and 0% in the ULD anti-S100 group). In assessing the effectiveness of anti motion sickness properties of drugs the special attention was paid to the possible impact of drugs on the stability of autonomic nervous system (ANS) in particular, shifting of the balance between its sympathetic and parasympathetic divisions. For this purpose, at each visit HRV parameters were analyzed at the rest condition and when performing the functional tests (breathing and orthostatic tests).

TABLE 2
Indexes of Halle's scale depending on applied preparation
after the performance of CCEAC test

| | Halle's scale (points) | |
|---|---|---|
| | Visit 1 | Visit 2 |
| | (one-day intake) | (course intake) |
| Preparation | (n = 15; M ± SE) | (n = 15; M ± SE) |
| ULD anti-S100 + | 12.00 ± 0.63 | 12.30 ± 0.59 |
| anti-eNOS | | |
| ULD anti-S100 | 13.30 ± 0.65 | 12.30 ± 0.46 |
| ULD anti-eNOS | 13.10 ± 0.78 | 12.00 ± 0.55 |
| Placebo | 13.40 ± 0.77 | 13.30 ± 0.45 |

TABLE 3
The dynamics of indexes of CCEAC test depending on applied preparation

| | Visit 1 (one-day intake) | | |
|---|---|---|---|
| | | Nystagmus | |
| | Tolerance of | time, sec. | Recovery time, |
| | CCEAC test, sec. | (n = 15; M ± | sec. (n = 15; M ± |
| Preparation | (n = 15; M ± SD) | SD) | SD) |
| ULD anti-S100 + | 104.10 ± 13.14 ** | 9.90 ± 1.20 * | 96.90 ± 13.54 *** |
| anti-eNOS | | | |
| ULD anti-S100 | 68.50 ± 6.57 ^{×} | 13.50 ± 1.51 | 194.20 ± 18.45 ^{×××} |
| ULD anti-eNOS | 75.00 ± 6.79 | 16.10 ± 1.68 | 202.50 ± 21.72 ^{×××} |
| Placebo | 61.30 ± 3.15 | 13.30 ± 1.12 | 241.70 ± 38.41 |
| P value on | 0.0182 | 0.0658 | 0.0001 |
| Kruskal-Wallis | | | |
| test ¹ | | | |

Visit 2 (course intake)

| | | | |
|---|---|---|---|
| ULD anti-S100 + | 134.70 ± 20.24 ** | 9.50 ± 1.38 ** | 117.90 ± 15.65 ** |
| anti-eNOS | | | |
| ULD anti-S100 | 82.70 ± 10.33 | 13.50 ± 1.69 | 167.50 ± 14.72 ^{×} |
| ULD anti-eNOS | 74.30 ± 9.49 ^{×} | 17.30 ± 2.40 ^{×××} | 209.20 ± 21.62 ^{××} |
| Placebo | 63.70 ± 3.91 | 15.00 ± 1.47 | 199.60 ± 31.19 |
| P value on | 0.0341 | 0.0244 | 0.0061 |
| Kruskal-Wallis | | | |
| test ¹ | | | |

Notes:
¹ for determination of significant difference between groups the Kruskal-Wallis test was used. If the test showed a significant difference of p < 0.05 for comparison between groups against each other the Mann-Whitney test was used.
* the significant difference in comparison with placebo, p < 0.05;
** the significant difference in comparison with placebo, p < 0.01;
*** the significant difference in comparison with placebo, p < 0.001.
^{×} the significant difference in comparison with ULD anti-S100 + anti-eNOS, p < 0.05;
^{××} the significant difference in comparison with ULD anti-S100 + anti-eNOS, p < 0.01;
^{×××} the significant difference in comparison with ULD anti-S100 + anti-eNOS, p < 0.001.

The analysis of HRV at the rest condition (in sitting position) before and after the CCEAC test (Table 4) detected that in subjects receiving study drugs had a tendency to an increased rate of SDNN indicating an increase in heart rate variability due to parasympathetic influence on heart rhythm. In response to a kinetic effect in all treatment groups the value of RMS-SD increased which characterizes the activity of the parasympathetic component of autonomic regulation. In the groups receiving the composition ULD anti-S100+anti-eNOS and ULD anti-S100 showed an increase in HF which also indicated a shift in autonomic balance toward parasympathetic link. Thus, after conducting CCEAC tests in all groups there was an increase of parasympathetic effects on heart rate.

TABLE 4
The HRV parameters of the study participants at rest
before and after the kinetic action

| | Visit 1 (one-day intake) | | Visit 2 (course intake) | |
|---|---|---|---|---|
| | After the | After the | After the drug | After the |
| Parameter | drug intake | CCEAC test | intake | CCEAC test |

ULD anti-S100 + anti-eNOS group (M ± SD)

| | | | | |
|---|---|---|---|---|
| SDNN, | 57.7 ± 5.51 | 68.2 ± 7.42 | 59.4 ± 5.03 | 65.6 ± 4.66 |
| msec. | | | | |
| RMSSD, | 43.1 ± 6.77 | 51.4 ± 9.22 | 47.0 ± 6.21 | 47.6 ± 5.33 |
| msec. | | | | |
| TP, msec. ² | 979.0 ± 186.06 | 1678.3 ± 397.11# | 1067.2 ± 167.24 | 1381.0 ± 166.30 |
| LF, msec. ² | 437.5 ± 709.6 | 709.6 ± 178.72 | 391.9 ± 75.61 | 588.5 ± 87.48 |
| HF, msec. ² | 171.5 ± 51.08 | 228.4 ± 76.79 | 206.5 ± 58.32 | 218.5 ± 43.96 |
| LF/HF, c.u. | 4.2 ± 0.82 | 4.9 ± 0.83 | 3.3 ± 0.83 | 4.2 ± 0.91 |

ULD anti-S100 group (M ± SD)

| | | | | |
|---|---|---|---|---|
| SDNN, | 60.9 ± 4.62 | 70.9 ± 5.90 | 59.1 ± 4.80 | 68.8 ± 4.87 |
| msec. | | | | |
| RMSSD, | 44.3 ± 5.39 | 50.6 ± 6.56 | 42.4 ± 4.63 | 47.8 ± 5.57 |
| msec. | | | | |
| TP, msec. ² | 832.2 ± 124.93* | 1342.8 ± 217.09 | 841.4 ± 149.93 | 1288.0 ± 163.52# |
| LF, msec. ² | 315.2 ± 52.38* | 550.9 ± 72.44# | 313.6 ± 66.71 | 540.7 ± 87.57# |
| HF, msec. ² | 151.4 ± 41.19 | 247.0 ± 69.53# | 138.3 ± 38.42 | 187.1 ± 39.80 |
| LF/HF, c.u. | 3.0 ± 0.54 | 4.0 ± 0.72 | 2.8 ± 0.53 | 4.0 ± 0.52 |

ULD anti-eNOS group (M ± SD)

| | | | | |
|---|---|---|---|---|
| SDNN, | 67.4 ± 7.73 | 78.6 ± 6.14 | 65.8 ± 8.68 | 69.0 ± 5.23 |
| msec. | | | | |
| RMSSD, | 53.0 ± 8.86 | 58.4 ± 7.68 | 59.6 ± 12.45 | 52.2 ± 5.30 |
| msec. | | | | |
| TP, msec. ² | 1307.8 ± 324.24 | 1841.1 ± 359.79# | 1232.3 ± 292.51 | 1275.4 ± 172.47 |
| LF, msec. ² | 576.5 ± 167.07 | 849.9 ± 194.2# | 527.2 ± 167.07 | 562.1 ± 89.38 |
| HF, msec. ² | 313.3 ± 139.90 | 285.3 ± 65.92 | 218.9 ± 74.78 | 216.3 ± 63.72 |
| LF/HF, c.u. | 3.6 ± 0.87 | 3.9 ± 0.82 | 3.7 ± 1.14 | 3.8 ± 0.58 |

Placebo group (M ± SD)

| | | | | |
|---|---|---|---|---|
| SDNN, | 64.6 ± 6.10 | 75.7 ± 6.42 | 61.1 ± 6.72 | 70.8 ± 6.79 |
| msec. | | | | |
| RMSSD, | 50.9 ± 7.74 | 53.1 ± 6.62 | 44.6 ± 6.63 | 44.3 ± 5.31 |
| msec. | | | | |
| TP, msec. ² | 1062.2 ± 150.02 | 1917.8 ± 318.96# | 898.8 ± 169.62 | 1418.5 ± 227.59# |
| LF, msec. ² | 440.6 ± 77.30 | 832.4 ± 181.15 | 334.8 ± 75.94 | 611.4 ± 113.64# |
| HF, msec. ² | 253.9 ± 59.95 | 266.7 ± 61.94 | 166.0 ± 48.14 | 174.1 ± 44.96 |
| LF/HF, c.u. | 3.4 ± 0.72 | 5.0 ± 1.33 | 3.4 ± 0.93 | 4.8 ± 0.83 |

Note:
*the significant difference in comparison with the placebo, p ≦ 0.05);
#the significant difference in comparison with baseline parameters, p ≦ 0.05.

The analysis of HRV in transition states showed that one-day intake of composition ULD anti-S100+anti-eNOS increased the reaction time (13.9±1.14; p≦0.05) and the stabilization time (24.2±1.28; p≦0.05) in comparison with the ULD anti-S100 and placebo (Table 5). The same factors exceeded the value of the placebo group and after the kinetic effect which demonstrated the positive effect of the combined drug on the reactivity of the ANS (increase of tolerance to changes in body position). The smallest difference between the maximum and minimum heart rate in the breath test (Table 6) confirmed a better balance of the two divisions of ANS after receiving a one-day composition ULD anti-S100+anti-eNOS (25.1±2.66 beats/min, p≦0.05). By the end of week course of therapy the stabilizing effect on the balance of ANS after the CCEAC test (with orthostatic and breath test) is also noticed in the group receiving the composition ULD anti-S100+anti-eNOS (Tables 5 and 6).

TABLE 5
The HRV parameters of participants of the study
at orthostatic test before and after kinetic action

| | Visit 1 (one-day intake) | | Visit 2 (course intake) | |
|---|---|---|---|---|
| | After drug | After CCEAC | After drug | After CCEAC |
| Parameter | intake | test | intake | test |

ULD anti-S100 + anti-eNOS (M ± SD) Group

| | | | | |
|---|---|---|---|---|
| Exercise | 1.30 ± 0.06 | 1.40 ± 0.04 | 1.30 ± 0.06 | 1.40 ± 0.06 |
| reaction, c.u. | | | | |
| Reaction time, | 13.9 ± 1.14*^{×} | 12.7 ± 1.24* | 11.8 ± 0.57 | 11.7 ± 1.09 |
| sec. | | | | |
| Stabilization | 24.2 ± 1.28*^{×} | 21.9 ± 1.44* | 20.6 ± 0.74 | 22.4 ± 1.44*^{×} |
| time, sec. | | | | |

ULD anti-S100 (M ± SD) Group

| | | | | |
|---|---|---|---|---|
| Exercise | 1.40 ± 0.04 | 1.30 ± 0.04 | 1.30 ± 0.04 | 1.30 ± 0.05 |
| reaction, c.u. | | | | |
| Reaction time, | 7.60 ± 1.05 | 10.6 ± 1.55 | 9.7 ± 1.21 | 10.0 ± 1.73 |
| sec. | | | | |
| Stabilization | 15.1 ± 1.16* | 18.3 ± 1.43 | 18.0 ± 1.18 | 18.0 ± 1.80 |
| time, sec. | | | | |

ULD anti-eNOS (M ± SD) Group

| | | | | |
|---|---|---|---|---|
| Exercise | 1.30 ± 0.04 | 1.30 ± 0.04 | 1.50 ± 0.12 | 1.30 ± 0.04 |
| reaction, c.u. | | | | |
| Reaction time, | 8.20 ± 0.94 | 9.10 ± 1.12 | 9.2 ± 0.77 | 8.3 ± 0.70 |
| sec. | | | | |
| Stabilization | 16.5 ± 1.02 | 17.1 ± 1.33 | 19.0 ± 2.04 | 16.7 ± 0.98 |
| time, sec. | | | | |

Placebo group (M ± SD)

| | | | | |
|---|---|---|---|---|
| Exercise | 1.30 ± 0.04 | 1.30 ± 0.04 | 1.40 ± 0.06 | 1.30 ± 0.06 |
| reaction, c.u. | | | | |
| Reaction time, | 9.5 ± 1.28 | 8.1 ± 0.90 | 10.4 ± 1.58 | 8.8 ± 1.09 |
| sec. | | | | |
| Stabilization | 18.3 ± 0.94 | 16.8 ± 1.09 | 18.0 ± 1.37 | 16.5 ± 1.11 |
| time, sec. | | | | |

Note:
*the significant difference in comparison with placebo, p ≦ 0.05);
^{×}the significant difference in comparison with ULD anti-S100, p ≦ 0.05.

TABLE 6
The HRV parameters of participants of the study
at breath test before and after kinetic action

| | Visit 1 (one-day intake) | | Visit 2 (course intake) | |
|---|---|---|---|---|
| | After drug | After CCEAC | After drug | After |
| Parameter | intake | test | intake | CCEAC test |

ULD anti-S100 + anti-eNOS (M ± SD) Group

| | | | | |
|---|---|---|---|---|
| Corellation | 1.5 ± 0.05* | 1.5 ± 0.06 | 1.5 ± 0.05 | 1.5 ± 0.05 |
| max HR/min | | | | |
| HR, c.u. | | | | |
| Difference | 25.1 ± 2.66* | 26.5 ± 2.77 | 26.5 ± 2.37 | 24.9 ± 2.24* |
| max HR-min | | | | |
| HR, beats/min. | | | | |

ULD anti-S100 (M ± SD) Group

| | | | | |
|---|---|---|---|---|
| Corellation | 1.5 ± 0.06 | 1.6 ± 0.05 | 1.5 ± 0.04 | 1.6 ± 0.06 |
| max HR/min | | | | |
| HR, c.u. | | | | |
| Difference | 27.7 ± 2.68 | 27.2 ± 2.40 | 25.7 ± 2.24 | 26.9 ± 2.67 |
| max HR-min | | | | |
| HR, beats/min. | | | | |

ULD anti-eNOS (M ± SD) Group

| | | | | |
|---|---|---|---|---|
| Corellation | 1.5 ± 0.05 | 1.5 ± 0.04 | 1.5 ± 0.06 | 1.6 ± 0.05 |
| max HR/min | | | | |
| HR, c.u. | | | | |
| Difference | 26.7 ± 2.44 | 26.2 ± 2.04 | 27.7 ± 2.47 | 27.3 ± 2.12 |
| max HR-min | | | | |
| HR, beats/min. | | | | |

Placebo group (M ± SD)

| | | | | |
|---|---|---|---|---|
| Corellation | 1.6 ± 0.07 | 1.6 ± 0.06 | 1.5 ± 0.05 | 1.6 ± 0.05 |
| max HR/min | | | | |
| HR, c.u. | | | | |
| Difference | 31.2 ± 3.06 | 28.2 ± 2.50 | 27.7 ± 2.37 | 29.2 ± 2.44 |
| max HR-min | | | | |
| HR, beats/min. | | | | |

Note:
*the significant difference in comparison with placebo, p ≦ 0.05

The results of self-esteem of functional state (well-being, activity, mood) of the subjects which was conducted by the participants of the study after the simulation of motion sickness (CCEAC tests) at the beginning and at the end of therapy showed that the subjects of all the groups have given ‘average’ points for each of the parameters (Table 7). Thus, on the background of drugs intake the CCEAC tolerance was satisfactory. The highest growth rates compared with data of the placebo group by the end of the 7^{th} day of intake (more than 10%) was observed in the group of composition of ULD anti-S100+anti-eNOS.

TABLE 7
The dynamics of parameters of self-esteem of functional
condition (well-being-activity-mood) of study participants

| | | |
|---|---|---|
| Parameter | Visit 1 (one-day intake) | Visit 2 (course intake) |

ULD anti-S100 + anti-eNOS (M ± SE) group

| | | |
|---|---|---|
| Well-being | 4.3 ± 0.26 | 4.6 ± 0.27 |
| Activity | 4.2 ± 0.20 | 4.2 ± 0.22 |
| Mood | 5.0 ± 0.16 | 5.2 ± 0.13 |

ULD anti-S100 (M ± SE) group

| | | |
|---|---|---|
| Well-being | 3.7 ± 0.21 | 4.3 ± 0.22 |
| Activity | 3.6 ± 0.17 | 4.0 ± 0.19 |
| Mood | 4.5 ± 0.16 | 4.9 ± 0.19 |

ULD anti-eNOS (M ± SE) Group

| | | |
|---|---|---|
| Well-being | 3.9 ± 0.25 | 4.1 ± 0.26 |
| Activity | 3.8 ± 0.25 | 3.9 ± 0.23 |
| Mood | 4.4 ± 0.19 | 4.6 ± 0.19 |

Placebo group (M ± SE)

| | | |
|---|---|---|
| Well-being | 4.0 ± 0.24 | 4.0 ± 0.24 |
| Activity | 3.8 ± 0.20 | 3.7 ± 0.26 |
| Mood | 4.3 ± 0.20 | 4.7 ± 0.24 |

The safety analysis included data from all the subjects who participated in the study. During the observation period a well tolerance of studied preparations was noticed. No adverse events associated with drug administration identified. All the subjects of studied groups completed treatment in the terms established by the study protocol; there was not persons early dropped out.

According to the results of physical examination including indicators of heart rate, systolic and diastolic blood pressure and according to the Harvard step test data the subjects were not recorded as with any abnormalities during the study (Table 8). All identified changes were not beyond the normal range. In this case, subjectively all subjects reported satisfactory well-being.

TABLE 8
The dynamics of physical parameters and exercise tolerance of study
participants before and after kinetic action

| | Visit 1 (one-day intake) | | Visit 2 (course intake) | |
|---|---|---|---|---|
| | After drug | After CCEAC | After drug | After |
| Parameter | intake | test | intake | CCEAC test |

ULD anti-S100 + anti-eNOS (M ± SE) Group

| | | | | |
|---|---|---|---|---|
| HR | 74.6 ± 3.36 | 68.4 ± 3.67 | 74.1 ± 3.10 | 67.7 ± 2.62 |
| (beats/min) | | | | |
| Systolic | 123.4 ± 2.83 | 125.9 ± 4.08 | 121.8 ± 2.65 | 128.3 ± 4.25 |
| blood | | | | |
| pressure | | | | |
| (mmhg.) | | | | |
| Diastolic | 74.0 ± 3.09 | 79.3 ± 2.62 | 76.2 ± 2.43 | 80.3 ± 3.30 |
| blood | | | | |
| pressure | | | | |
| (mmhg.) | | | | |
| Step-test | — | 53.6 ± 2.60 | — | 52.3 ± 2.09 |
| index | | | | |

ULD anti-S100 (M ± SE) Group

| | | | | |
|---|---|---|---|---|
| HR | 73.5 ± 2.57 | 69.7 ± 2.78 | 72.1 ± 2.84 | 67.7 ± 2.39 |
| (beats/min) | | | | |
| Systolic | 127.5 ± 2.55 | 133.5 ± 4.77 | 127.1 ± 2.55 | 129.9 ± 5.06 |
| blood | | | | |
| pressure | | | | |
| (mmhg.) | | | | |
| Diastolic | 75.5 ± 2.65 | 82.6 ± 3.31 | 74.9 ± 2.41 | 82.3 ± 3.19 |
| blood | | | | |
| pressure | | | | |
| (mmhg.) | | | | |
| Step-test | — | 50.6 ± 1.71 | — | 53.0 ± 1.63 |
| index | | | | |

ULD anti-eNOS (M ± SE) Group

| | | | | |
|---|---|---|---|---|
| HR | 76.5 ± 2.59 | 67.3 ± 1.98 | 77.3 ± 2.02 | 70.1 ± 3.23 |
| (beats/min) | | | | |
| Systolic | 127.3 ± 3.14 | 131.5 ± 5.16 | 123.5 ± 3.06 | 129.3 ± 4.13 |
| blood | | | | |
| pressure | | | | |
| (mmhg.) | | | | |
| Diastolic | 75.2 ± 2.24 | 80.3 ± 2.66 | 73.9 ± 2.83 | 81.0 ± 3.22 |
| blood | | | | |
| pressure | | | | |
| (mmhg.) | | | | |
| Step-test | — | 51.8 ± 2.12 | — | 51.2 ± 2.21 |
| index | | | | |

Placebo group (M ± SE)

| | | | | |
|---|---|---|---|---|
| HR | 74.5 ± 2.78 | 68.9 ± 3.46 | 73.9 ± 3.23 | 72.3 ± 3.58 |
| (beats/min) | | | | |
| Systolic | 125.3 ± 3.30 | 133.3 ± 4.73 | 124.3 ± 2.83 | 126.9 ± 3.95 |
| blood | | | | |
| pressure | | | | |
| (mmhg.) | | | | |
| Diastolic | 76.2 ± 2.15 | 81.7 ± 2.83 | 75.4 ± 1.86 | 79.7 ± 3.03 |
| blood | | | | |
| pressure | | | | |
| (mmhg.) | | | | |
| Step-test | — | 50.0 ± 2.03 | — | 50.1 ± 1.99 |
| index | | | | |

In addition to the hemodynamic parameters, for evaluation of the safety of studied drugs and its possible negative impact on the central nervous functions, the following physiological parameters were examined in subjects: (RMO (reaction on moving object), SMRT (simple motor reaction time), RA (range of attention), attention span (AS), and attention stability factor (ASF)). In addition, the Stange's test was conducted to assess tolerance to hypoxia.

According to received results (Table 9) neither one-day or course drug intake had a significant effect on the estimated parameters. Indexes of sensory motor coordination (SMRT, RMO) did not differ from the results of the placebo group before and after the CCEAC test at both visits. Study data of such complicated functions like volume and stability of attention showed that the studied drugs both before and after the CCEAC test did not change the degree of concentration and shift in attention not being different from the placebo group.

The analysis of standard exercise tests with breath holding showed a tendency to increase of the tolerance of hypoxia by the subjects (Table 9). When holding the breath the duration of Stange's test grew after taking all study drugs. However, only intake of the combination composition ULD anti-S100+anti-eNOS showed significantly longer time in the holding of the breath after the kinetic effect (68.1±18.8 sec. at baseline and 91.7±27.4 sec. after the CCEAC test; p<0.05). The increase of tolerance of hypoxia was also noted when the Gench's test (Stange's test) (breath holding at expiration, P>0.05) was used.

TABLE 9
The dynamics of parameters of psycho-physiological
state of study participants before and after kinetic action

| | Visit 1 (one-day intake) | | Visit 2 (course intake) | |
|---|---|---|---|---|
| | After drug | After CCEAC | After drug | After CCEAC |
| Parameter | intake | test | intake | test |

ULD anti-S100 + anti-eNOS (M ± SE) Group

| | | | | |
|---|---|---|---|---|
| SMRT | 257.5 ± 8.67 | 268.9 ± 10.18 | 269.6 ± 9.75 | 279.9 ± 12.24 |
| RMO, c.u. | 50.1 ± 3.92 | 49.5 ± 4.50 | 47.3 ± 4.86 | 47.0 ± 3.54 |
| RMO, % of | 3.0 ± 0.95 | 4.5 ± 1.15 | 5.3 ± 1.58 | 4.0 ± 1.11 |
| target hit | | | | |
| AS, sec. | 5.2 ± 0.34 | 5.2 ± 0.35 | 5.2 ± 0.41 | 5.1 ± 0.40 |
| Range of | 41.7 ± 2.36 | 39.9 ± 2.38 | 38.1 ± 2.17 | 37.5 ± 2.04 |
| attention, | | | | |
| sec. | | | | |
| ASF | 17.4 ± 1.66 | 17.2 ± 1.51 | 18.0 ± 1.71 | 18.8 ± 1.72 |
| Stange's test | 68.1 ± 4.85 | 91.7 ± 7.07* | 71.8 ± 6.02 | 85.5 ± 9.36 |
| Gench's test | 47.1 ± 4.03 | 50.1 ± 3.94 | 46.7 ± 3.28 | 48.1 ± 4.52 |

ULD anti-S100 (M ± SE) Group

| | | | | |
|---|---|---|---|---|
| SMRT | 258.9 ± 9.95 | 282.4 ± 13.56 | 268.4 ± 11.37 | 279.1 ± 9.20 |
| RMO, c.u. | 58.1 ± 6.40 | 57.5 ± 6.34 | 55.1 ± 05.06 | 53.8 ± 5.02 |
| RMO, % of | 3.7 ± 1.50 | 2.0 ± 0.82 | 2.3 ± 0.83 | 5.0 ± 1.69 |
| target hit | | | | |
| AS, sec. | 6.0 ± 0.40 | 6.4 ± 0.52 | 6.2 ± 0.42 | 6.0 ± 0.41 |
| Range of | 42.6 ± 2.68 | 42.1 ± 2.27 | 42.7 ± 2.30 | 41.9 ± 2.52 |
| attention, | | | | |
| sec. | | | | |
| ASF | 14.5 ± 1.16 | 14.9 ± 1.26 | 15.3 ± 1.13 | 15.4 ± 1.18 |
| Stange's test | 59.0 ± 4.09 | 72.6 ± 6.19 | 64.5 ± 4.93 | 75.9 ± 5.67 |
| Gench's test | 47.1 ± 4.48 | 49.4 ± 4.69 | 48.3 ± 4.30 | 48.8 ± 4.14 |

ULD anti-eNOS (M ± SE) group

| | | | | |
|---|---|---|---|---|
| SMRT | 257.7 ± 8.49 | 279.4 ± 14.23 | 266.7 ± 13.19 | 275.5 ± 11.44 |
| RMO, c.u. | 48.3 ± 3.67 | 51.9 ± 4.39 | 52.5 ± 4.79 | 49.6 ± 4.22 |
| RMO, % of | 2.3 ± 0.83 | 2.0 ± 0.82 | 3.3 ± 1.26 | 5.7 ± 1.68 |
| target hit | | | | |
| AS, sec. | 5.9 ± 0.25 | 6.0 ± 0.34 | 5.5 ± 0.24 | 5.9 ± 0.33 |
| Range of | 41.9 ± 2.10 | 43.8 ± 2.39 | 41.3 ± 2.00 | 42.5 ± 2.22 |
| attention, | | | | |
| sec. | | | | |
| ASF | 13.7 ± 1.34 | 14.8 ± 1.31 | 15.6 ± 1.24 | 14.1 ± 1.40 |
| Stange's test | 62.5 ± 5.49 | 69.5 ± 5.09 | 56.7 ± 3.34 | 73.1 ± 7.98 |
| Gench's test | 43.1 ± 3.51 | 45.7 ± 3.15 | 43.4 ± 3.77 | 45.8 ± 4.03 |

Placebo group (M ± SE)

| | | | | |
|---|---|---|---|---|
| SMRT | 267.6 ± 7.64 | 290.1 ± 11.33 | 281.1 ± 9.78 | 263.3 ± 6.85 |
| RMO, c.u. | 60.7 ± 8.31 | 54.1 ± 5.57 | 51.1 ± 3.69 | 52.6 ± 5.38 |
| RMO, % of | 3.7 ± 1.03 | 3.7 ± 1.24 | 3.3 ± 0.93 | 4.3 ± 1.61 |
| target hit | | | | |
| AS, sec. | 6.1 ± 0.71 | 5.7 ± 0.36 | 5.5 ± 0.32 | 5.9 ± 0.71 |
| Range of | 41.9 ± 2.09 | 42.4 ± 2.81 | 41.3 ± 2.18 | 39.6 ± 2.26 |
| attention, | | | | |
| sec. | | | | |
| ASF | 14.5 ± 1.64 | 14.5 ± 1.79 | 15.3 ± 1.55 | 15.9 ± 1.58 |
| Stange's test | 63.7 ± 4.71 | 67.9 ± 6.90 | 64.8 ± 5.94 | 83.0 ± 12.24 |
| Gench's test | 44.7 ± 2.52 | 47.1 ± 3.30 | 43.7 ± 2.71 | 47.8 ± 3.78 |

Thus, the study using an experimental motion sickness demonstrated the effectiveness of the combination composition ULD anti-S100+anti-eNOS and monocomponent preparation ULD-S100. The studied drugs increase the stability of the subjects to the kinetic effect after simulation of the clinical and physiological effects of motion sickness contributing to more mild clinical process of motion sickness and earlier recovery of the subjects after cessation of treatment. In addition, it was shown that the anti motion sickness effect of the combination composition (compositions ULD anti-S100+anti-eNOS) increases the efficiency of individual components. The effectiveness of the combination composition ULD anti-S100+anti-eNOS in the control of the vestibular-autonomic and sensory reactions of a body in experimental motion sickness increases at course intake. It should be noted that ULD anti-eNOS in the form of monopreparation does not have a protective effect against motion sickness but when combined with ULD anti-S100 significantly enhances the anti motion sickness effect of the last one which manifests itself as at one-day so at short course intake of the drug. The best ability to adjust the transient processes that is to influence to the reactivity of the parasympathetic and sympathetic parts of CNS as well as adaptive capabilities of ANS in a state of motion sickness (to increase the tolerance to sudden changes in a body position) was observed in the composition ULD anti-S100+anti-eNOS which is an important component of anti motion sickness properties of the drug. Composition ULD anti-S100+anti-eNOS and monocomponent preparation ULD anti-S100 when using them as anti motion sickness preparation including when performing an operator functions are safe and do not adversely impact on the physical and psychophysiological parameters.

Combination composition ULD anti-S100+anti-eNOS and ULD anti-S100 can be recommended for the prophylaxis and relief of kinesia in motion disease (including sea, air and car sicknesses) to persons with low and moderate degree of stability. The combination composition has high safety and no adverse effects on the quality of professional activity.

### Example 3

Tablets weighing 300 mg were used to assess efficacy of the treatment of subjects with vegetative dysfunction syndrome (VDS) of psychophysiological and hormonal imbalance origin with the combination pharmaceutical composition ULD anti-S100+anti-eNOS and ULD anti-S100. The tablets were saturated with pharmaceutical composition containing water-alcoholic solutions (6 mg/tablet) of activated-potentiated forms of polyclonal affinity purified rabbit antibodies to brain-specific protein S-100 (anti-S100) and endothelial NO-synthase (anti-eNOS) in ultralow doses (ULD) obtained by ultradilution of the starting stock solution (with concentration 2.5 mg/mL) by 100¹², 100³⁰, 100²⁰⁰ times equivalent to the mixture of centennial homeopathic dilutions C12, C30, C200 (ULD of anti-S100+anti-eNOS).

The reference group included the subjects receiving tablets weighing 300 mg saturated with water-alcoholic solution (3 mg/tablet) of activated-potentiated form of polyclonal rabbit antibodies to brain-specific protein S-100, purified on antigen, in ultralow dose (ULD of anti-S100) obtained by ultradilution the starting stock solution (concentration 2.5 mg/mL) by 100¹², 100³⁰, 100²⁰⁰ times, equivalent to the mixture of centennial homeopathic dilutions C12, C30, C200.

The study design was monocenter open-label randomized comparative clinical study of efficacy and safety of drugs containing ULD of anti-S100+anti-eNOS and ULD of anti-S100 as monotherapy, when treating subjects with vegetative dysfunction syndrome (VDS) of psychophysiological and hormonal imbalance origin.

The study enrolled 12 subjects with VDS of psychophysiological origin and VDS of hormonal imbalance origin, aged 23-61 years. Mean age of the subjects was 49.25±12.63 years.

After confirmation of the subject's compliance with inclusion and exclusion criteria the subjects were randomized into one of the study groups: Group I—ULD of anti-S100+anti-eNOS group, included 6 subjects (3 subjects with VSD of psychophysiological origin and 3 subjects with VDS of hormonal imbalance origin). The mean age of group I was 41.33±12.5 years (17.7% males and 82.3% females); Group 2—ULD of anti-S100 group, included 6 subjects (3 subjects with VSD of psychophysiological and 3 subjects with VDS of hormonal imbalance origin). The mean age of group 2 subjects was 57.16±4.35 years (17.7% males and 82.3% females).

Four visits to the study site were made during this study. Treatment stage lasted from Visit 1 to Visit 3. Visit 3 (Day 56±5) was the first study endpoint, after which the follow-up stage was started. Follow-up stage lasted till Visit 4 (Day 84±5).

Safety analysis included the data of all subjects enrolled into the study (n=12). During the entire observation period subjects demonstrated good drug tolerability. No adverse events were reported. One subject did not attend Visit 2 and was not included into analysis. All other study subjects completed the treatment within the terms established by the study protocol. No subject who withdrew from the study ahead of the term has been registered.

Assessment of effect of ULD of anti-S100+anti-eNOS on the main symptoms of VDS as well as anxiety and depressive disorders (Beck Depression questionnaire) revealed improved quality of life of the subjects demonstrated as statistically significant increase in the total SF-36 questionnaire score (subscale “physical health” from 38.04±2.44 to 47.84±1.27, p=0.005, subscale “mental health”—from 57.88±3.94 to 72.75±1.64, p<0.01) as well statistically significant reduction of the total score of Beck Depression questionnaire (from 11.0±1.4 to 5.5±1.37, p<0.02)

Assessment of effect of ULD of anti-S100 on the main VDS symptoms as well as anxiety and depressive disorders (Beck Depression questionnaire) revealed improved quality of life demonstrated as statistically significant increase in the total SF-36 questionnaire score (subscale “physical health” from 56.107±1.36 to 70.7±1.39, p<0.001). No tendency for increased total score of “physical health” subscale in this group was reported.

Analysis of changes in anxiety and depressive disorders in ULD of anti-S100 groups revealed statistically significant reduction of the total score of Beck Depression questionnaire (from 10.5±1.04 to 5.33±1.5, p<0.02) (Table 10).

TABLE 10

| | SF-36 (physical | SF-36 (mental | Beck Depression |
|---|---|---|---|
| | health) | health) | Questionnaire |
| ULD of anti- | 38.04 ± 2.44 | 57.88 ± 3.94 | 11.0 ± 1.4 |
| S100 + anti-eNOS | | | |
| prior to treatment | | | |
| ULD of anti- | 47.84 ± 1.27* | 72.75 ± 1.64** | 5.5 ± 1.37*** |
| S100 + anti-eNOS | | | |
| after treatment | | | |
| ULD of anti-S100 | 46.99 ± 8.09 | 56.107 ± 1.36 | 10.5 ± 1.04 |
| prior to treatment | | | |
| ULD of anti-S100 | 49.17 ± 2.68 | 70.7 ± 1.39**** | 5.33 ± 1.5*** |
| after treatment | | | |

*p vs. the baseline = 0.005
**p vs. the baseline < 0.01
***p vs. the baseline < 0.02
****p vs. the baseline < 0.001

Significant intergroup differences in these parameters after treatment have not been determined. During the planning of the study and enrolment of the subjects the groups were divided into the following subgroups:

1. subjects with Vegetative Dysfunction Syndrome of psychophysiological origin (chronic stress) who were to receive ULD of anti-S100+anti-eNOS as monotherapy;

2. subjects with Vegetative Dysfunction Syndrome of psychophysiological origin (chronic stress), who were to receive ULD of anti-S100 as monotherapy;

3. subjects with Vegetative Dysfunction Syndrome of hormonal imbalance (menopausal) origin who were to receive ULD of anti-S100+anti-eNOS as monotherapy;

4. subjects with Vegetative Dysfunction Syndrome of hormonal imbalance (menopausal) origin who were to receive ULD of anti-S100 S100 as monotherapy.

Subgroup tendencies in data analysis corresponded to the ones in general group analysis, though they were less significant that was probably associated with small number of observations (Table 11, 12).

TABLE 11
VDS of Hormonal Imbalance (menopausal) origin

| | SF-36 | SF-36 | Beck Depression |
|---|---|---|---|
| | (physical health) | (mental health) | Questionnaire |
| ULD of anti- | 38.5 ± 2.99 | 57.9 ± 4.42 | 11.0 ± 2.0 |
| S100 + anti-eNOS | | | |
| prior to treatment | | | |
| ULD of anti- | 47.99 ± 1.48* | 72.75 ± 1.85* | 5.33 ± 0.57*** |
| S100+ anti-eNOS | | | |
| after treatment | | | |
| ULD of anti-S100 | 47.39 ± 8.35 | 56.79 ± 1.23 | 10.0 ± 1.0 |
| prior to treatment | | | |
| ULD of anti-S100 | 48.96 ± 3.16 | 70.71 ± 1.68** | 4.66 ± 0.057**** |
| after treatment | | | |

*p vs. baseline < 0.05
**p vs. baseline < 0.005
***p vs. baseline = 0.053
****p vs. baseline = 0.01

TABLE 12
VDS of Hormonal Imbalance (chronic stress) origin

| | SF-36 (physical | SF-36 (mental | Beck Depression |
|---|---|---|---|
| | health) | health) | Questionnaire |
| ULD of anti- | 37.57 ± 2.31 | 57.85 ± 4.39 | 11.0 ± 1.0 |
| S100 + anti-eNOS | | | |
| prior to treatment | | | |
| ULD of anti- | 47.69 ± 1.32* | 72.73 ± 1.82** | 5.66 ± 2.08**** |
| S100 + anti-eNOS | | | |
| after treatment | | | |
| ULD of anti-S100 | 47.39 ± 8.35 | 55.42 ± 1.31 | 11.0 ± 1.0 |
| prior to treatment | | | |
| ULD of anti-S100 | 48.96 ± 3.16 | 70.69 ± 1.65*** | 6.0 ± 2.0**** |
| after treatment | | | |

*p vs. baseline < 0.02
**p vs. baseline < 0.05
***p vs. baseline = 0.002
****p vs. baseline = 0.082

Intergroup and intragroup analysis of changes in arterial pressure, integrative vegetative parameters, and variation pulsometry values indicated no statistically significant tendencies, except for reduced Vegetative Balance Index (VBI). Most probably, this is associated with inadequate number of observations.

VBI is an integrative parameter calculated as Mo amplitude (number of cardiointervals corresponding to mode range) and Variation range (difference between maximal and minimal R-R values) ratio. Reduction of this parameter evidences displacement of vegetative balance from sympathicotonia to normo- and vagotonia, i.e. enhanced effect of parasympathetic segments of vegetative nervous system (VNS).

In the hormonal imbalance VDS group a statistically significant tendency for reduced VBI was noted in ULD of anti-S100+anti-eNOS subgroup. A statistically significant (p<0.05) difference between ULD of anti-S100+anti-eNOS and ULD of anti-S100 subgroups has been noted (Table 13).

TABLE 13
Hormonal Imbalance VDS

| | VBI prior to | VBI after |
|---|---|---|
| | treatment | treatment |
| ULD of anti-S100 + | 721.1 ± 38.52 | 416.86 ± 73.72*# |
| anti-eNOS | | |
| ULD of anti-S100 | 735.4 ± 58.42 | 696.26 ± 61.85 |

*p vs. baseline < 0.05
#p vs. ULD of anti-S100. < 0.05

Therefore, the clinical study of the combination pharmaceutical composition ULD of anti-S100+anti-eNOS demonstrated positive effect on the quality of life of subjects with Vegetative Dysfunction Syndrome (VDS) of psycho-physiological and hormonal imbalance origin, positive effect on anxiety and depressive disorders of subjects. Positive effect of the combination pharmaceutical composition of the present invention on vegetative nervous system has been registered. Furthermore, high tolerability of the combination pharmaceutical composition of the present invention was noted. No adverse events have been reported.

### Example 4

Alzheimer's disease (AD) is a neurodegenerative disease that is characterized by lowering of cognitive functions, memory deterioration, confused consciousness, and emotional changes. Although the main cause of this pathology is nowadays considered the accumulation of beta amyloid which leads to the formation of beta-amyloid plaques and neurofibrillary tangles in brain tissues; AD is also accompanied by a deficiency of cholinergic system. This is the basis of a most common way of modeling of AD in animals with the help of antagonist of cholinergic system of scopolamine. Injection of scopolamine into experimental animals (usually rats or mice) interrupts the ability to learn and leads to deterioration of memory.

Various methods were used to assess cognitive functions of rats and mice, including Morris water maze. The essence of this test is that the animals are released into a container with cloudy water from different points are forced to look for a hidden fixed platform. The advantage of this method is that it allows the researcher to monitor the process of animal training (the formation of ideas about the spatial alignment of the platform no matter where the animal was placed in the water) so as to assess the memory strength (for this the test is conducted when the platform is removed).

The effectiveness in rats with Scopolamine amnesia of the combination pharmaceutical composition of the present invention containing activated-potentiated forms of polyclonal affinity purified on antigen of rabbit brain-specific proteins S-100 (anti-S100) and to endothelial NO-synthase (anti-eNOS) in ultra low doses (ULD) obtained by super dilution of storage stock solution (with concentration of 2.5 mg/ml) in 100¹², 100³⁰, 100²⁰⁰ times, equivalent to centesimal homeopathic dilutions C12, C30, C200 (ULD anti-S100+anti-eNOS) is studied.

In a study of the effectiveness of the drug ULD anti-S100+anti-eNOS in rats with scopolamine amnesia (a model of Alzheimer's disease) 48 male rats of the Rj: Wistar (Han) line (weight 180-280 g) were used. During 4 days the rats were subdermally injecting with normal saline (n=12, intact) or scopolamine in doze of 0.5 mg/kg (n=36) (scopolamine-induced amnesia). Rats with scopolamine-induced amnesia were divided into three groups and administered with distilled water (7.5 ml/kg, n=12, control group 1), or ULD anti-S100 (7.5 ml/kg, n=12, group 2) or ULD anti-S100+anti-eNOS (7.5 ml/kg, n=12, group 3) intragastrically for 9 days (4 days prior to the injection of scopolamine, 4 days against the background of scopolamine and 1 day after the last scopolamine injection).

The training session in the Morris water maze was conducted within 4 days of the scopolamine injection through 60 minutes after administration of tested drugs and 30 minutes after administration of scopolamine (4 sequential tests at interval of 60 seconds). Morris' maze is a round reservoir (diameter—150 cm, height—45 cm) at 30 cm filled with water (26-28° C.). At 18 cm from the edge of the container there is hidden platform (diameter—15 cm) buried on 1.5 cm below the water level. Cloudy water made by adding a non-toxic dye (e.g., milk powder) makes the platform invisible. For each test the animal was placed in a maze in one of the initial points that are equidistant from the hidden platform and the animal was allowed to find the platform. If the animal could not find the platform within 120 seconds, the animal was put on the platform and left for 60 seconds and the test was restarted. During the four tests in random order the animals began to walk through the maze twice from each starting point. The tests were recorded on videotape and then analyzed for distance overcomes searching the platform in each trial and the latent period of searching for the platform. On day 5 the test was performed: the platform was removed from the maze and rats were given free float for 60 seconds. The time spent in the place where the platform used to be was recorded.

The administration of scopolamine significantly worsened the ability of animals to learn. In the control group the time spent by animals searching for platforms and the distance that animals swam searching for the platform, significantly increased (Table 14, 15). The test shows that the memory of animals in the control group worsened: the animals in this group spent less time in the place where the platform used to be located than intact animals (Table 16). The administration of ULD anti-S100 didn't lead to improvement of the studied parameters (Tables 14, 15, 16). The administration of ULD anti-S100+anti-eNOS led to some improvement in learning which resulted in a shortening of the latent time of the platform search time (Table 14) and covered distance (Table 15) within 4 days of training and an improvement of memory as reflected in increase of the time spent in a place where the platform used to be located (Table 16).

TABLE 14
Latent period of the platform search, sec

| | Training | | | |
|---|---|---|---|---|
| Group | 1^{st} day | 2^{nd} day | 3^{rd} day | 4^{th} day |
| Intact, n = 12 | 54.7 ± 6.2 | 30.8 ± 2.8 | 26.9 ± 5.1 | 20.5 ± 3.6 |
| Control, n = 12 | 100.1 ± 6.8*** | 92.4 ± 9.3*** | 81.4 ± 10.7*** | 77.7 ± 9.4*** |
| ULD anti-S100, n = 12 | 106.8 ± 7.0 | 99.3 ± 7.8 | 95.6 ± 9.0 | 80.4 ± 11.1 |
| ULD anti-S100 + anti-eNOS, | 94.4 ± 7.2 | 90.7 ± 8.2 | 78.3 ± 8.6 | 60.1 ± 10.2 |
| n = 12 | | | | |

***difference from intact is significant, p < 0.05

TABLE 15
Distance overcome to search the platform, cm

| | Training | | | |
|---|---|---|---|---|
| Group | 1^{st} day | 2^{nd} day | 3^{rd} day | 4^{th} day |
| Intact, n = 12 | 1055.7 ± 94.6 | 659.5 ± 62.2 | 564.8 ± 119.3 | 406.1 ± 61.2 |
| Control, n = 12 | 2587.1 ± 217.2*** | 2559.6 ± 250.5*** | 2397.9 ± 312.6 | 2366.1 ± 293.8*** |
| ULD anti- | 2797.2 ± 208.9 | 2865.2 ± 255.1 | 2857.0 ± 300.8 | 2457.4 ± 344.4 |
| S100, n = 12 | | | | |
| ULD anti- | 2434.3 ± 222.8 | 2529.9 ± 282.7 | 2344.2 ± 283.0 | 1905.1 ± 343.7 |
| S100 + anti- | | | | |
| eNOS, n = 12 | | | | |

***difference from intact is significant, p < 0.05

TABLE 16
Time spent in a place where the platform used to be located, sec.

| | Test | | |
|---|---|---|---|
| Group | 0-30 sec. | 30-60 sec. | 0-60 sec. |
| Intact, n = 12 | 40.8 ± 4.1 | 36.8 ± 3.6 | 38.5 ± 2.6 |
| Control, n = 12 | 18.4 ± 2.8*** | 18.8 ± 1.9*** | 18.8 ± 1.7*** |
| ULD anti-S100, n = 12 | 13.3 ± 2.1 | 21.5 ± 2.6 | 17.6 ± 1.3 |
| ULD anti-S100 + | 19.1 ± 4.8 | 23.8 ± 2.2 | 21.2 ± 2.5 |
| anti-eNOS, n = 12 | | | |

***difference from intact is significant, p < 0.05

Thus, in model of Alzheimer's disease, the administration of the complex ULD anti-S100+anti-eNOS was more effective in comparison with administration of ULD anti-S100 and vehicle.

What is claimed is:

1. A combination pharmaceutical composition comprising a) an activated-potentiated form of an antibody to brain-specific protein S-100 and b) activated-potentiated form of an antibody to endothelial NO synthase.

2. The combination pharmaceutical composition of claim 1, wherein the activated-potentiated form of an antibody to brain-specific protein S-100 is to the entire bovine brain-specific protein S-100.

3. The combination pharmaceutical composition of claim 1, wherein the activated-potentiated form of an antibody to brain-specific protein S-100 is to brain-specific protein S-100 having SEQ ID NO: 9, SEQ ID NO: 10, SEQ ID NO: 11, or SEQ ID NO: 12.

4. The combination pharmaceutical composition of claim 1, wherein the activated-potentiated form of an antibody to endothelial NO synthase is to the entire bovine NO synthase.

5. The combination pharmaceutical composition of claim 1, wherein the activated-potentiated form of an antibody to endothelial NO synthase is to the entire human NO synthase.

6. The combination pharmaceutical composition of claim 1, wherein the activated-potentiated form of an antibody to brain-specific protein S-100 is in the form of a mixture of C12, C30, and C50 homeopathic dilutions impregnated onto a solid carrier and the activated-potentiated form of an antibody to endothelial NO synthase is in the form of mixture of C12, C30, and C50 homeopathic dilutions impregnated onto the solid carrier.

7. The combination pharmaceutical composition of claim 1, wherein the activated-potentiated form of an antibody to brain-specific protein S-100 is in the form of a mixture of C12, C30, and C200 homeopathic dilutions impregnated onto a solid carrier and the activated-potentiated form of an antibody to endothelial NO synthase is in the form of mixture of C12, C30, and C200 homeopathic dilutions impregnated onto the solid carrier.

8. The combination pharmaceutical composition of claim 1, wherein the activated-potentiated form of an antibody to endothelial NO synthase is in the form of mixture of C12, C30, and C50 homeopathic dilutions impregnated onto a solid carrier and the activated-potentiated form of an antibody to brain-specific protein S-100 is in the form of mixture of C12, C30, and C50 homeopathic dilutions impregnated onto the solid carrier.

9. The combination pharmaceutical composition of claim 1, wherein the activated-potentiated form of an antibody to endothelial NO synthase is in the form of mixture of C12, C30, and C200 homeopathic dilutions impregnated onto a solid carrier and the activated-potentiated form of an antibody to brain-specific protein S-100 is in the form of mixture of C12, C30, and C200 homeopathic dilutions impregnated onto the solid carrier.

10. The combination pharmaceutical composition of claim 1, wherein the activated-potentiated form of an antibody to brain-specific protein S-100 is a monoclonal, polyclonal or natural antibody.

11. The combination pharmaceutical composition of claim 10, wherein the activated-potentiated form of an antibody to brain-specific protein S-100 is a polyclonal antibody.

12. The combination pharmaceutical composition of claim 1, wherein the activated-potentiated form of an antibody to brain-specific protein S-100 is prepared by successive centesimal dilutions coupled with shaking of every dilution.

13. The combination pharmaceutical composition of claim 1, wherein the activated-potentiated form of an antibody to endothelial NO synthase is a monoclonal, polyclonal or natural antibody.

14. The combination pharmaceutical composition of claim 13, wherein the activated-potentiated form of an antibody to endothelial NO synthase is a polyclonal antibody.

15. The combination pharmaceutical composition of claim 1, wherein the activated-potentiated form of an antibody to endothelial NO synthase is prepared by successive centesimal dilutions coupled with shaking of every dilution.

16. A method of treating vertigo of various genesis, kinetosis and vegetative-vascular dystonia by administration of the combination pharmaceutical composition of claim 1.

17. A method of reducing kinetosis as measured by the CCEAC test by administration of the combination pharmaceutical composition of claim 1.

18. A method of stabilizing the effect on the imbalance of autonomic nervous system as measured by the CCEAC test by administration of the combination pharmaceutical composition of claim 1.

19. The method of claims 16-18, wherein the combination pharmaceutical composition is administered in one to two unit dosage forms, each of the dosage form being administered from once daily to four times daily.

20. The method of claim 19, wherein the combination pharmaceutical composition is administered in one to two unit dosage forms, each of the dosage form being administered twice daily.
